(19) United States
(12) Patent Application Publication
(10) Pub. No.: US 2019/0105332 A1
(43) Pub. Date: Apr. 11, 2019

(54) METHODS OF TREATING PROSTATE CANCER

(71) Applicant: Janssen Oncology, Inc., Los Angeles, CA (US)

(72) Inventor: Namphuong Tran, Los Angeles, CA (US)

(21) Appl. No.: 15/891,974

(22) Filed: Feb. 8, 2018

Related U.S. Application Data
(60) Provisional application No. 62/570,781, filed on Oct. 11, 2017.

(51) Int. Cl. A61K 31/58; A61K 31/573; A61P 35/04; G16H 10/60

(52) U.S. Cl. CPC A61K 31/58; G16H 10/60; A61P 35/04; A61K 31/573

(57) ABSTRACT

Disclosed are methods of treating prostate cancer by administering abiraterone acetate plus prednisone with androgen deprivation therapy.

### CROSS-REFERENCE TO RELATED APPLICATIONS

This application claims the benefit of U.S. Provisional Application No. 62/570,781, filed on Oct. 11, 2017, which is incorporated by reference herein in it's entirety.

### FIELD OF THE INVENTION

The present invention relates to the treatment of metastatic hormone-naïve prostate cancer (“mHNPC”) in a subject by administering to such subject abiraterone acetate plus prednisone with androgen deprivation therapy (“ADT”). Also disclosed are methods of selling or offering for sale an abiraterone acetate drug product.

### BACKGROUND OF THE INVENTION

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens, such as testosterone, promote prostate cancer tumor growth. Not all prostate cancer is the same. It ranges from cancer confined to the prostate gland to cancer that has spread outside of the prostate to the lymph nodes, bones, or other parts of the body. The extent or spread of prostate cancer determines the stage. At its early stages, localized prostate cancer is often treated with, local therapy including, for example, surgical removal of the prostate gland and radiotherapy. However, when local therapy fails to cure prostate cancer, as it does in up to a third of men, the disease progresses into incurable metastatic disease (i.e., disease in which the cancer has spread from one part of the body to other parts).

Historically, ADT has been the standard of care for patients with metastatic prostate cancer. ADT is often very effective at shrinking or slowing the growth of prostate cancer that has spread. Patients with newly diagnosed mHNPC (the same patient population can also be referred to as having metastatic castration-sensitive prostate cancer), particularly with high-risk characteristics, have a poor prognosis. While the majority of patients initially start on ADT, it usually becomes less effective over time.

The clinical benefit of adding docetaxel (a chemotherapeutic agent) to ADT versus ADT alone in treating patients with mHNPC has been demonstrated in three randomized phase 3 trials. These are: Chemohormonal Therapy Versus Androgen Ablation Randomized Trial for Extensive Disease in Prostate Cancer (CHAARTED), Systemic Therapy in Advancing or Metastatic Prostate Cancer; Evaluation of Drag Efficacy (STAMPEDE), and GETUG-15. These studies collectively included more than 3000 men with metastatic, castration-sensitive prostate cancer. For men eligible for chemotherapy, ADT plus docetaxel is now a standard of care for metastatic, castration-sensitive prostate cancer. But there are barriers to using docetaxel, including advanced patient age, poor performance status, coexisting illnesses and patient preferences. Also, chemotherapy-related toxicity and related complications may be a concern. Deaths associated with docetaxel related toxicity were documented in ail three randomized trials where docetaxel was added to ADT.

Abiraterone acetate, a prodrug of abiraterone, inhibits 17α hydroxylase/C17, 20-lyase (cytochrome P450c17 [CYP17]), a key enzyme in androgen biosynthesis. Abiraterone acetate in combination with prednisone has been approved for the treatment of men with metastatic castration-resistant prostate cancer (“mCRPC”) who have received prior chemotherapy containing docetaxel. The efficacy and safety of abiraterone acetate (1,000 mg daily tablet dose) and prednisone (5 mg twice daily) therapy inpatients with mCRPC was established by the results of COU-AA-301 and COU-AA-302, both Phase 3, multinational, randomized, double-Mind, placebo-controlled studies. Study COU-AA-301 was the first Phase 3 study to demonstrate that further lowering testosterone concentrations below that achieved with ADT using CYP17 inhibition with abiraterone acetate improves survival in patients with mCRPC. COU-AA-302 demonstrated significantly improved overall survival (“OS”) and radiographic progression-tree survival (“rPFS”) in chemotherapy-naïve patients with mCRPC treated, with abiraterone -acetate plus prednisone compared with placebo plus prednisone.

For patients with localized prostate cancer at high, risk for metastasis after treatment, two randomized studies were conducted with men receiving neoadjuvant therapy. Men with high-risk or intermediate risk localized prostatic cancer were treated using abiraterone acetate and prednisone in addition to ADT prior to undergoing prostatectomy. There were higher rates of PSA response and higher rates of complete pathologic response or near-complete pathologic response in these patients compared to those with only ADT or with ADT and delayed treatment with abiraterone acetate and prednisone. Not all patient populations respond to the same therapies equally, but this invited research concerning a potential role for inhibiting extra gonadal androgen biosynthesis, before castration resistance emerges, in different patient populations.

Notwithstanding the available treatments, alternative treatments for safe and effective treatment of men with mHNPC are needed.

### SUMMARY OF THE INVENTION

The present invention is directed to a method for treating mHNPC in a human in need of such treatment comprising, consisting of, and/or consisting essentially of administering to the human a safe and efficacious amount of abiraterone acetate, a safe and efficacious amount of prednisone and a safe and efficacious amount of ADT.

In one embodiment, tire present invention is directed to a method of treating metastatic hormone-naive prostate cancer in a human comprising, consisting of and/or consisting essentially of adding a safe and effective amount of abiraterone acetate and a sale and effective amount of prednisone to ADT. ADT may include an orchiectomy, prior to or during treatment, or a hormonal ablation agent. In a preferred embodiment, the method comprises, consists of and/or consists essentially of administering to the human about 1000 mg/day of abiraterone acetate or a pharmaceutically acceptable salt thereof and about 5 mg/day of prednisone.

In one embodiment, the invention is directed, to a method of treating a human with newly diagnosed metastatic castration-sensitive prostate cancer comprising, consisting of and/or consisting essentially of administering a combination therapy demonstrated to increase overall survival of men with newly diagnosed metastatic castration-sensitive prostate cancer, wherein the treatment comprises, consists of and/or consists essentially of safe and effective amounts of abiraterone acetate, prednisone and androgen deprivation therapy.

In one embodiment, the invention is directed to a method of treating a human with newly diagnosed metastatic castration-sensitive prostate cancer comprising, consisting of and/or consisting essentially of administering a combination therapy demonstrated to increase radiographic progression-free survival of men with newly diagnosed metastatic castration-sensitive prostate cancer, wherein the treatment comprises, consists of, and/or consists essentially of safe and effective amounts of abiraterone acetate, prednisone and androgen deprivation therapy.

In one embodiment, the invention is directed to a method of treating a human with newly diagnosed metastatic castration-sensitive prostate cancer comprising, consisting of and/or consists essentially of administering a combination therapy demonstrated to increase time to pain progression for men with newly diagnosed metastatic castration-sensitive prostate cancer, wherein the treatment comprises, consists of, and/or consists essentially of safe and effective amounts of abiraterone acetate, prednisone and androgen deprivation therapy.

In one embodiment, the invention is directed to a method of treating a human with newly diagnosed metastatic castration-sensitive prostate cancer comprising, consisting of, and/or consisting essentially of administering a combination therapy demonstrated to increase time to a next symptomatic skeletal event for men with newly diagnosed metastatic castration-sensitive prostate cancer, wherein the treatment comprises, consists of, and/or consists essentially of safe and effective amounts of abiraterone acetate, prednisone and androgen deprivation therapy.

In one embodiment, the invention is directed to a method of treating a human with newly diagnosed metastatic castration-sensitive prostate cancer comprising, consisting of, and/or consisting essentially of administering a combination therapy demonstrated to increase time to time to PSA progression for men with newly diagnosed metastatic castration-sensitive prostate cancer, wherein the treatment comprises, consists of and/or consists essentially of safe and effective amounts of abiraterone acetate, prednisone and androgen deprivation therapy. In still further aspects, described herein are methods of offering for sale an anti-androgen comprising, consisting of, or consisting essentially of offering to place the apalutamide into the stream of commerce whereto said anti-androgen includes a package insert that contains instructions for safely and effectively treating prostate cancer using the anti-androgen. In certain embodiments, the anti-androgen is apalutamide

In certain embodiments, the invention is directed to a method of selling an approved drug product comprising, consisting of and/or consisting essentially of abiraterone acetate, said method comprising, consisting of and/or consisting essentially of a sale of such drag product, wherein a label for a reference listed drug for such drug product includes instructions for treating metastatic castration-sensitive prostate cancer. In other embodiments, the drug product is an ANDA drug product or a supplemental New Drug Application drug product. In another aspect, in the case of apalutamide, the label for said reference listed drug includes a daily dose of 1000 mg of abiraterone acetate and 5 mg prednisone.

In certain embodiments, the invention is directed to a method of offering for sale a drug product comprising, consisting of and/or consisting essentially of abiraterone acetate, said method comprising, consisting of and/or consisting essentially of offering for sale of such drug product, wherein a label for a reference listed drug for such drug product includes instructions for treating metastatic castration-sensitive prostate cancer, in other embodiments, the drug product is an ANDA drug product or a supplemental New Drug Application drug product.

### BRIEF DESCRIPTION OF THE DRAWINGS

FIG. 1A depicts Kaplan-Meier estimates of overall survival.

FIG. 1B depicts Kaplan-Meier estimates of radiographic progression-free survival.

FIG. 1C shows overall survival subgroups.

FIG. 1D shows radiographic progression free survival subgroups.

In FIGS. 1A-1D, the dashed lines indicate the median; CI denotes confidence interval.

FIG. 2A depicts secondary efficacy end points on pain progression.

FIG. 2B depicts secondary efficacy end points on PSA progression.

FIG. 2C depicts secondary efficacy end points on symptomatic skeletal events.

FIG. 2D depicts secondary efficacy end points on initiation of cytotoxic chemotherapy.

FIG. 2E depicts secondary efficacy end points on subsequent prostate cancer therapy.

FIG. 3 depicts the baseline demographic and disease characteristics between the study groups.

FIG. 4 depicts Kaplan-Meier overall survival corves in COU-AA-301.

FIG. 5 depicts Kaplan-Meier overall survival curves in COU-AA-302

FIG. 6 depicts Kaplan-Meier without radiographic progression-free survival in COU-AA-302.

FIG. 7 depicts Kaplan-Meier plot of overall survival; intent to treat population in LATITUDE.

### DETAILED DESCRIPTION OF THE INVENTION

Broadly, the present invention is directed to a method for treating mHNPC in a human in need of such treatment, where such treatment includes administering a safe and efficacious amount of abiraterone acetate and a safe and efficacious amount of prednisone with ADT.

Men with newly diagnosed mHNPC can have variable outcomes The LATITUDE study was the first study to explore the utility of more effective blockade of the androgen receptor axis with the addition of abiraterone acetate plus prednisone to ADT in men with mHNPC. This study enrolled those considered to have high-risk feature as defined by the presence of 2 or more of the following poor prognostic features: Gleason score ≥8, presence of ≥3 bone lesions, or visceral metastasis, which are associated with poor survival. In addition, 50% of patients enrolled had symptomatic disease at baseline. The patient population appears similar to high-burden disease populations in the three randomized trials testing ADT plus docetaxel as the outcomes of the control arms (i.e., ADT alone) were similar across these studies.

The efficacy of ADT-abiraterone acetate-prednisone reported herein, with a 38% reduction in the risk of death, compares favorably with previous findings. The data demonstrate that more effective up-front inhibition of androgen receptor signaling in patients with castration-naïve prostate cancer leads to improved outcomes. Of note, early use of abiraterone acetate plus prednisone resulted in improved survival even if more patients from the ADT-placebos group received life-prolonging treatments after progression.

This study demonstrated that adding abiraterone acetate and low-dose prednisone (5 mg prednisone) to ADT is superior to ADT alone in treating subjects with mHNPC, especially those subjects with high-risk prognostic factors. The significant clinical benefits of adding abiraterone acetate and prednisone to ADT may include longer overall survival, longer radiographic progression-free survival, longer time to pain progression, longer time to a next symptomatic skeletal event, a longer time to PSA progression, and/or a longer time to subsequent therapy, as compared to treatment with ADT alone.

In this phase 3 study involving men with high-risk, newly diagnosed mHNPC, ADT plus abiraterone and prednisone significantly improved overall survival, with a reduction in the risk of death of 38% (hazard ratio, 0.62), The addition of abiraterone plus prednisone to ADT also significantly improved radiographic progression-free survival (hazard ratio, 0.4) and all secondary endpoints defined in the trial. These findings led to the unanimous recommendation by the independent data monitoring committee to unblind the study and allow crossover to ADT-abiraterone-prednisone. With the statistical significance at the first interim analysis for overall survival, this analysis is considered “final.”

The magnitude of the clinic al benefits in this patient population, including the duration and effect of tins combination therapy, was unexpected. The difference in expected overall survival as compared to actual overall survival was statistically significant (as measured using Harvard ratio).

The clinical benefit observed here contrasts with that from the many previous attempts with castration and “first-generation” androgen receptor inhibitors, which only showed a small improvement with a combined androgen blockade approach, presumably owing to their lowered potency and partial agonist activity. The addition, of abiraterone acetate plus prednisone to ADT provides significant clinical benefit.

The overall safety profile of ADT-abiraterone acetate-prednisone was consistent with prior studies inpatients with metastatic castration-resistant prostate cancer. The observed degrees of hypertension and hypokalemia, both medically manageable, only rarely requiring treatment discontinuation, and seldom leading to significant consequences, point to the need for proper and timely management.

The present invention is directed to a method for treating mHNPC in a human in need of such treatment comprising, consisting of and/or consisting essentially of administering to the human a safe and efficacious amount of abiraterone acetate, a safe and efficacious amount of prednisone and a safe and efficacious amount of ADT.

“ADT” includes surgical castration (orchiectomy) and/or the administration of luteinizing hormone-releasing hormone (“LHRH”) agonists to a human. Examples of LHRH agonists include goserelin acetate, histrelin acetate, leuprolide acetate, and triptorelin palmoate. Physicians can prescribe LHRH agonists in accordance with instructions, recommendations and practices. This may include about 0.01 mg to about 20 mg of goserelin over a period of about 28 days to about 3 months, preferably about 3.6 mg to about 10.8 mg of goserelin over a period of about 28 days to about 3 months; about 0.01 mg to about 200 mg of leuprolide over a period of about 3 days to about 12 months, preferably about 3.6 mg of leuprolide over a period of about 3 days to about 12 months; or about 0.01 mg to about 20 mg of triptorelin over a period of about 1 month, preferably about 3.75 mg of triptorelin over a period of 1 month. About 50 mg of histrelin acetate over a period of 12 months of histrelin acetate or about 50 μg per day of histrelin acetate.

The term “composition” refers to a pharmaceutical product that includes the specified ingredients sometimes in safe and effective amounts, as well as any product that results, directly, or indirectly, from combinations of the specified ingredients in the specified amounts.

The term “hormone-naïve prostate cancer” or “HNPC” refers to a stage of the disease when the subjects have not yet received hormone therapy or ADT. HNPC is further categorized into biochemical recurrence (in which subjects have a rising prostate-specific antigen (“PSA”) after treatment, but the tumor is still localized).

The term “metastatic prostate cancer” refers to the form of prostate cancer in which the cancer has spread or metastasized to other parts of the body.

The term “mHNPC” means metastatic hormone-naïve prostate cancer where subjects with high-risk metastatic hormone-naïve prostate cancer have at least two of the following factors: Gleason score of eight or above (a grading system used to evaluate the prognosis of someone with prostate cancer), presence of three or more lesions on a bone scan, or presence of measurable visceral metastasis (spread to other organs) on CT or MRI, excluding lymph node disease.

The term “pharmaceutically acceptable” as used herein pertains to active pharmaceutical ingredient, materials, compositions and dosage forms that are, within the scope of sound medical judgment, suitable for use in contact with the tissues of a subject without excessive toxicity, irritations, allergic response, or other problem or complication, commensurate with a reasonable benefit/risk ratio. Each carrier, excipient, etc. must all be “acceptable” in the sense of being compatible with the other ingredients of the formulation.

The term “safe and effective amount” refers to an amount of an active pharmaceutical ingredient that elicits the desired biological or medicinal response in a subject's biological system without the risks outweighing the benefits of such response in accordance with the Federal Food, Drug, and Cosmetic Act, as amended (secs, 201-902, 52 Stat. 1040 et seq., as amended; 21 U.S.C. §§ 321-392). Safety is often measured by toxicity testing to determine the highest tolerable dose or the optimal dose of an active pharmaceutical ingredient needed to achieve the desired benefit. Studies that look at safety also seek to identify any potential adverse effects that may result from exposure to the drug. Efficacy is often measured by determining whether an active pharmaceutical ingredient demonstrates a health benefit over a placebo or other intervention when tested in an appropriate situation, such as a tightly controlled clinical trial.

The term “subject” refers to a human.

The term “treatment” refers to the treatment of a subject afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

The term, “drug product” is product that contains an active pharmaceutical ingredient that has been approved for marketing by a governmental authority, e.g., the Food and Drug Administration or the similar authority in other countries.

The term “Reference Listed Drug (RLD)” is a drug product to which new generic versions are compared to show that they are bioequivalent. It is also a medicinal product that has been granted marketing authorization by a Member State of the European Onion or by the Commission on the basis of a completed dossier, i.e., with the submission of quality, pre-clinical and clinical data in accordance with Articles 8 (3), 10a, 10b or 10c of Directive 2001/83/EC and to which the application for marketing authorization for a generic/hybrid medicinal product refers, by demonstration of bioequivalence, usually through the submission of the appropriate bioavailability studies.

In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the proposed generic drug product is the same as the RLD in certain ways. Specifically, with limited exceptions, a drug product for which an ANDA is submitted must have, among other things, the same active ingredients), conditions of use, route of administration, dosage form, strength, and (with certain permissible differences) labeling as the RLD. The RLD is the listed drag to which the ANDA applicant must show its proposed ANDA drug product is the same with respect to active ingredients), dosage form, route of administration, strength, labeling, and conditions of use, among other characteristics. In the electronic Orange Book, there will is a column for RLDs and a column, for reference standards. In the printed version of the Orange Book, the RLDs and reference standards are identified by specific symbol.

In Europe, Applicants identify in the application form for its generic/hybrid medicinal product, which is the same as a ANDA or sNDA drug product, the reference medicinal product (product name, strength, pharmaceutical form, MAH, first authorization, Member State/Community), which is synonymous with a RLD, as follows:

1. The medicinal product that is or has been authorized in the EEA, used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.

2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.

3. The medicinal product (product name, strength, pharmaceutical form, MAH, Member State of source) used for the bioequivalence study(ies) (where applicable).

The terms “sale” or “selling” means transferring a drug product, e.g., a pharmaceutical composition or an oral dosage form, from a seller to a buyer.

The term “offering for sale” means the proposal of a sale by a seller to a buyer for a drug product, e.g., a pharmaceutical composition and an oral dosage form.

The treatment may be administered by at least one oral dosage form, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a preferable amount of abiraterone acetate administered in accordance with the present invention is in the range of about 250 to about 1,250 mg/day. More preferably it is between about 500 to about 1,000 mg/day and most preferably about 1,000 mg/day. Abiraterone acetate is preferably administered one time per day. It may be recommended to take abiraterone acetate on an empty stomach; at least 2 hours before and at least 1 hour after eating.

In general, a preferable amount of prednisone administered in accordance with the present invention is in the range of about 5 to about 10 mg/day. Most preferably about 5 mg/day. The prednisone can be administered one time per day or in divided doses two times per day.

### EXAMPLES

The following Examples are set forth to aid in the understanding of the invention, and are not intended and should not be construed to limit in any way the invention set forth in the claims which follow thereafter.

### Example 1

A protocol was developed for a multinational, randomized, double-blind, active-controlled study designed to determine if newly diagnosed subjects with mHNPC who have high-risk prognostic factors will benefit from the addition of abiraterone acetate and low-dose prednisone to ADT, The study was referred to as the LATITUDE study. A total of 1199 patients were randomized from Feb. 12, 2013, through Dec. 11, 2014, to ADT-abiraterone acetate-prednisone (n=597) or ADT-placebos (n=602)

The study population included newly diagnosed (within 3 months prior to randomization) adult men with high-risk mHNPC Subjects were stratified by presence of visceral disease (yes/no) and Eastern Cooperative Oncology Group (ECOG) performance grade (0, 1, versus 2) prior to randomization. Subjects had to have distant metastatic disease as documented by positive bone scan or metastatic lesions on computed tomography (CT) or magnetic resonance imaging (MRI) to be eligible.

More specifically, eligible patients (aged ≥18 years and with Eastern Cooperative Oncology Group [ECOG] performance status score of 0 to 2) were newly diagnosed (within 3 months prior to randomization) with pathologically confirmed diagnosis of prostate cancer without neuroendocrine differentiation, or small-ceil histology. Patients had high-risk, metastatic hormone-sensitive prostate cancer documented by positive bone scan or metastatic lessons at the tune of diagnosis on computed tomography (CT) or magnetic resonance imaging (MRI) according to Response Valuation Criteria in Solid tumors (RECIST) version 1.1, and had to have at least two of the three following high-risk factors associated with poor prognosis: Gleason score ≥8, ≥3 bone lesions, presence of measurable visceral metastases. Prior pharmacotherapy, radiation therapy, or surgery for metastatic prostate cancer were exclusion criteria, with the exception of ≤3 months of androgen deprivation therapy (ADT) with luteinizing hormone-releasing hormone analogues or orchiectomy with or without concurrent first-generation androgen receptor antagonists prior to baseline, no more than one course of palliative radiation or surgical therapy to treat metastatic disease symptoms prior to randomization.

Eligible subjects may have received ADT (LHRH agonist or antagonist) or had an orchiectomy within 3 months of randomization. Upon entering the study, medical ADT could have been continued with the use of an LHRH agonist or orchiectomy. For subjects receiving an LHRH agonist, the use of an anti-androgen was allowed, for a maximum of 2 weeks after Cycle 1 Day 1. Subjects could have elected to undergo an orchiectomy instead of medical ADT with an LHRH agonist during the study.

If the LHRH agonist was not started until subjects were randomized into the study, then investigators could initiate an anti-androgen shortly before or at the start of an LHRH agonist and continue its use for at least 7 days and up to 2 weeks after the start of an LHRH agonist. This is to address the tumor flare that could be associated with the initiation of LHRH agonist. The continued use of anti-androgens beyond the first 2 weeks after Cycle 1 Day 1 was prohibited.

The study included a Screening Phase of up to 28 days before randomization to establish study eligibility and document baseline measurements; a Double-blind Treatment Phase; and a Follow-up Phase of up to 60 months to monitor survival status and subsequent prostate cancer therapy. Each cycle was 28 days. Treatment continued until disease progression, withdrawal of consent or the occurrence of unacceptable toxicity. In the event of a positive study result (efficacy boundary was crossed) at either of the interim analyses and the sponsor's decision was made to allow the study to proceed into the Open-label Extension (OLE) Phase, all subjects participating in the Double-blind Treatment Phase had the opportunity to enroll in an OLE Phase of this protocol. The OLE Phase allowed subjects to receive active drug (abiraterone acetate plus prednisone) until the Long-term Extension (LTE) Phase. In the event of a positive study result at final analysis and the sponsor's notification to initiate the LTE Phase, subjects who were still receiving abiraterone acetate plus prednisone could continue to receive treatment in the LTE Phase for an additional period of up to 3 years.

Subjects who met all of the inclusion criteria and none of the exclusion criteria were centrally randomized in a 1:1 ratio to receive ADT and abiraterone acetate (1000 mg orally daily as four 250 mg tablets) and prednisone (5 mg daily) (ADT-abiraterone-prednisone group) or ADT and placebos (ADT-placebos or control group).

Selection of the LHRH agonist was by investigator's choice provided that the dosing (dose and frequency of administration) was consistent with prescribing information. Each subject was reviewed by the sponsor before randomization to ensure that select eligibility criteria have been met.

Patients were stratified by presence or absence of measurable visceral disease and ECOG performance status grade (0 to 1 vs. 2). Abiraterone or placebo was administered at least 1 hour before and 2 hours after a meal All patients, who had not undergone surgical castration, received ongoing ADT to achieve or maintain a serum testosterone level of <50 ng per deciliter (1.7 nmol per liter). Safety and dosing compliance were evaluated during each study visit, at treatment discontinuation, if applicable, and at the end-of-study visit.

Abiraterone acetate/placebo was to be taken on an empty stomach. No food should have been consumed for at least 2 hours before the dose of abiraterone acetate/placebo and for at least 1 hour after the dose of abiraterone acetate/placebo was taken. If an abiraterone acetate or placebo dose was missed, it was to be omitted and not made up.

Subjects were monitored for safety throughout the study. Adverse events including laboratory adverse events were graded and summarized using National Cancer Institute Common Terminology Criteria for Adverse Events (NCI-CTCAE), Version 4.0. Dose modifications were made as required according to dose modification rules. An Independent Data Monitoring Committee (IDMC) was commissioned, for the study to perform regular review of the safety data and all data review at the planned interim analyses.

### Example 2

For the LATITUDE study described in Example 1, the co-primary efficacy end points were overall survival and radiographic progression-free survival. Overall survival was defined as the time from randomization to death from any cause, and radiographic progression-free survival as the time from randomization to the occurrence of radiographic progression or death from any cause. Radiographic progression of soft tissue lesions was evaluated by either CT or MRI on the basis of RECIST version 1.1. Progression on bone scan was assessed by adaptation of Prostate Cancer Working Group 2 (PCWG2) criteria, as follows:
Patients who had ≥2 new bone lesions on 16-week scans, with confirmatory scans performed 6 or more weeks later
   Patients with confirmatory scans that showed ≥2 new lesions compared with 16-week scans (i.e., total of four new lesions compared with baseline scan) were considered, to have disease progression by bone scan
   Patients who on confirmatory scans did not show ≥2 new lesions compared with 16-week scans were not considered to have disease progression at that time. All subsequent scans served as “confirmatory scans” and were considered to show disease progression with >2 new lesions compared with 16-week scans
Patients who did not have ≥2 new bone lesions on 16-week scans did not require a confirmatory scan. For any subsequent scan beyond 16 weeks, the first scan that would show ≥2 new lesions compared with baseline scans was considered to demonstrate disease progression by bone scan

Potential bias favoring the ADT-abiraterone-prednisone group in radiographic progression assessment by investigators was audited based on the PhRMA method in a sample of 202 randomly selected patients with blinded central review. Pre-specified secondary end points were time to next “skeletal-related event” (currently called “symptomatic skeletal event,” i.e., either clinical/pathologic fracture, spinal cord compression, palliative radiation to bone, or surgery to bone), time to prostate-specific antigen (PSA) progression by PCWG2 criteria, time to next subsequent therapy for prostate cancer, time to initiation of chemotherapy, and time to pain progression.

### Example 3

For the LATITUDE study described in Example 1, the overall level of significance was 0.05, with allocation between the co-primary end points of radiographic progression-free survival (0.001) and overall survival (0.049). One analysis was performed for radiographic progression-free survival when approximately 565 progression-free events were observed, which provides a statistical power of 94% to detect a hazard ratio of 0.667 at a two-tailed significance level of 0.001. For overall survival, approximately 852 events were required at the final analysis to detect a hazard ratio of 0.81 at a two-tailed significance level of 0.049, with a statistical power of 85%. Two interim analyses were included.

TABLE 1

| Overall Assumption | Rpfs | OS |
|---|---|---|
| A | 0.001 | 0.049 |
| Power | 94% | 85% |
| HR | 0.67 | 0.81 |
| Expected events | 565 (single | 426, 554, 852 |
| | analysis) | (two interim, one |
| | | final analysis |

| | Interim 1* | Interim 2 | |
|---|---|---|---|
| | (50% of total | (65% of total | |
| Planned OS Analysis | events) | events) | Final |
| Projected observed OS | ~426 | ~554 | ~852 |
| events | | | |
| Efficacy boundary (HR) | 0.78 | 0.81 | 0.87 |
| Stopping P value under Hₒ | (0.011) | (0.022) | (0.049) |
| (cumulative alpha spend) | | | |

*At time of final radiographic progression-free analysis. Hazard ratio (HR) denotes hazard ratio, H₀ no improvement, rPFS radiographic progression-free survival, and OS overall survival.

### Example 4

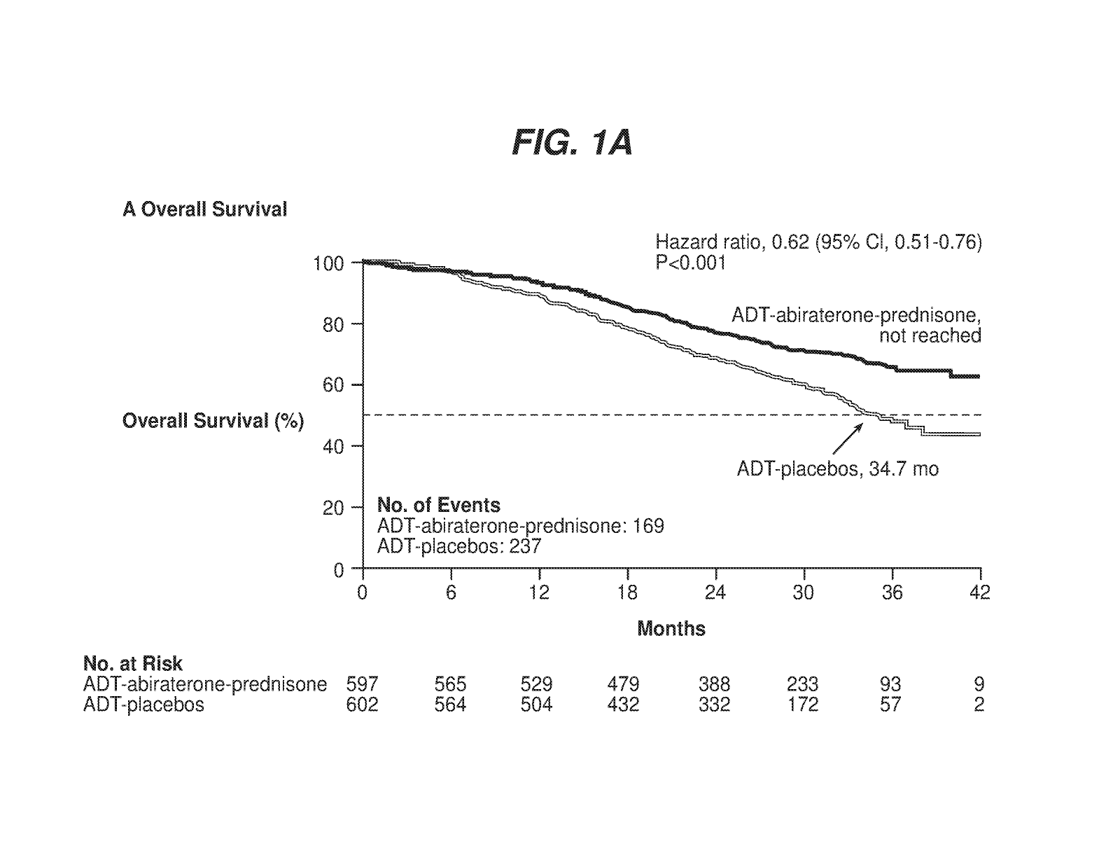

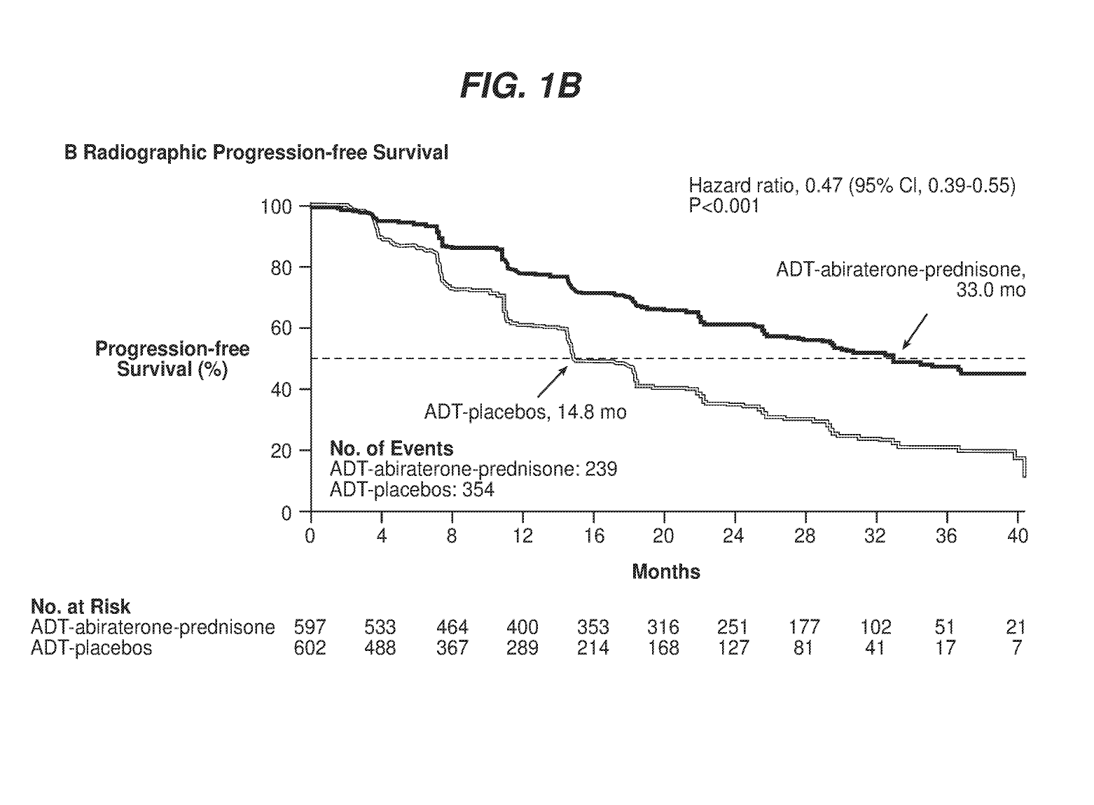

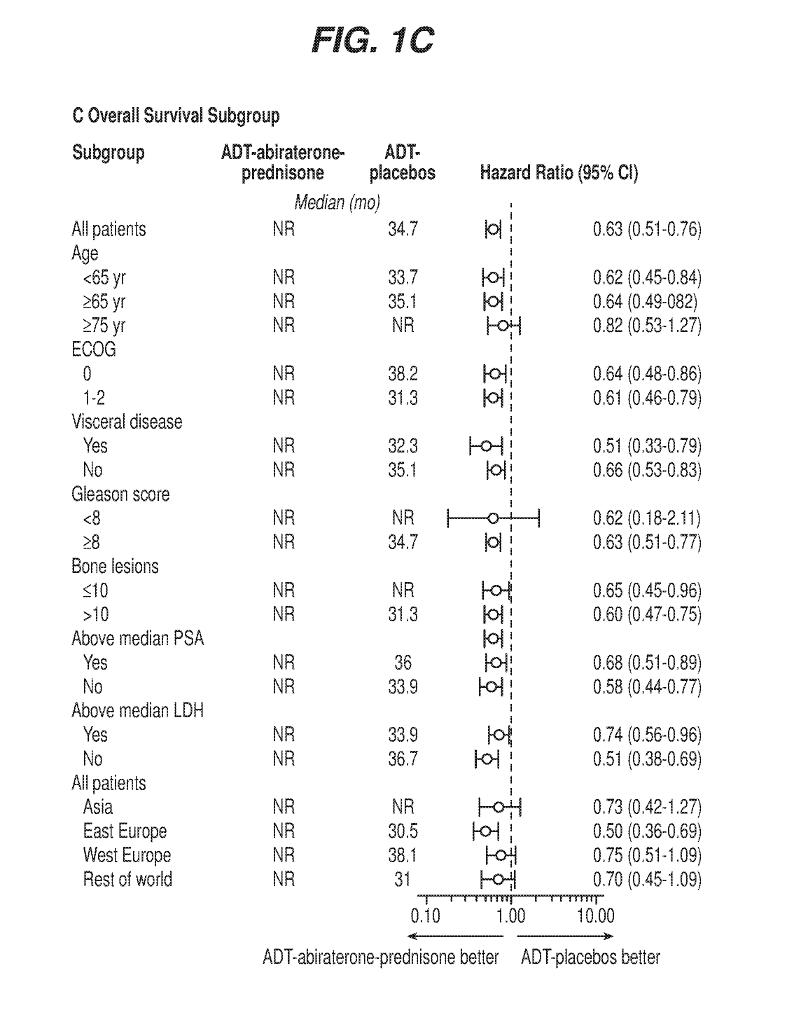

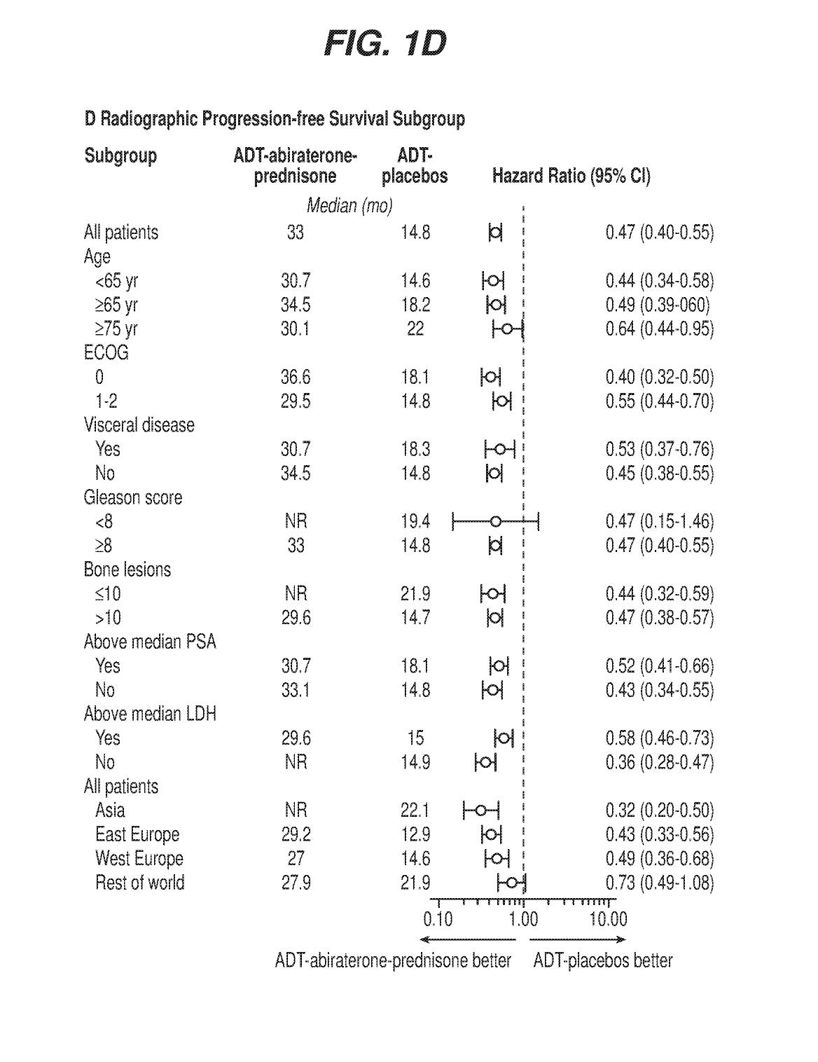

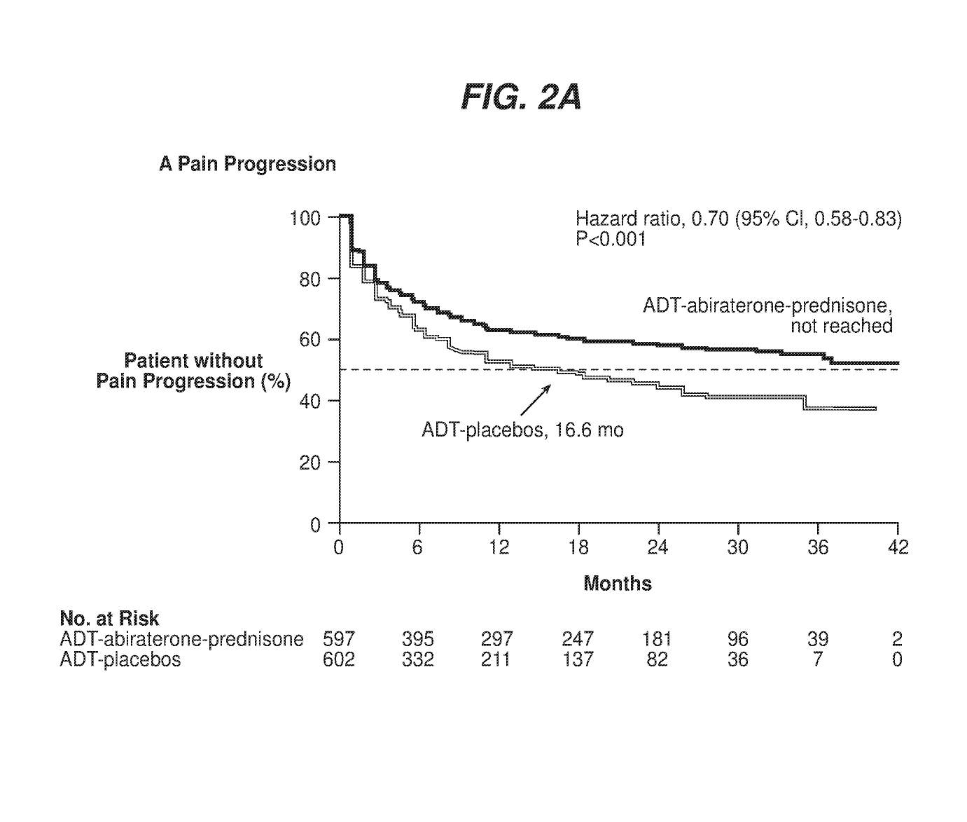

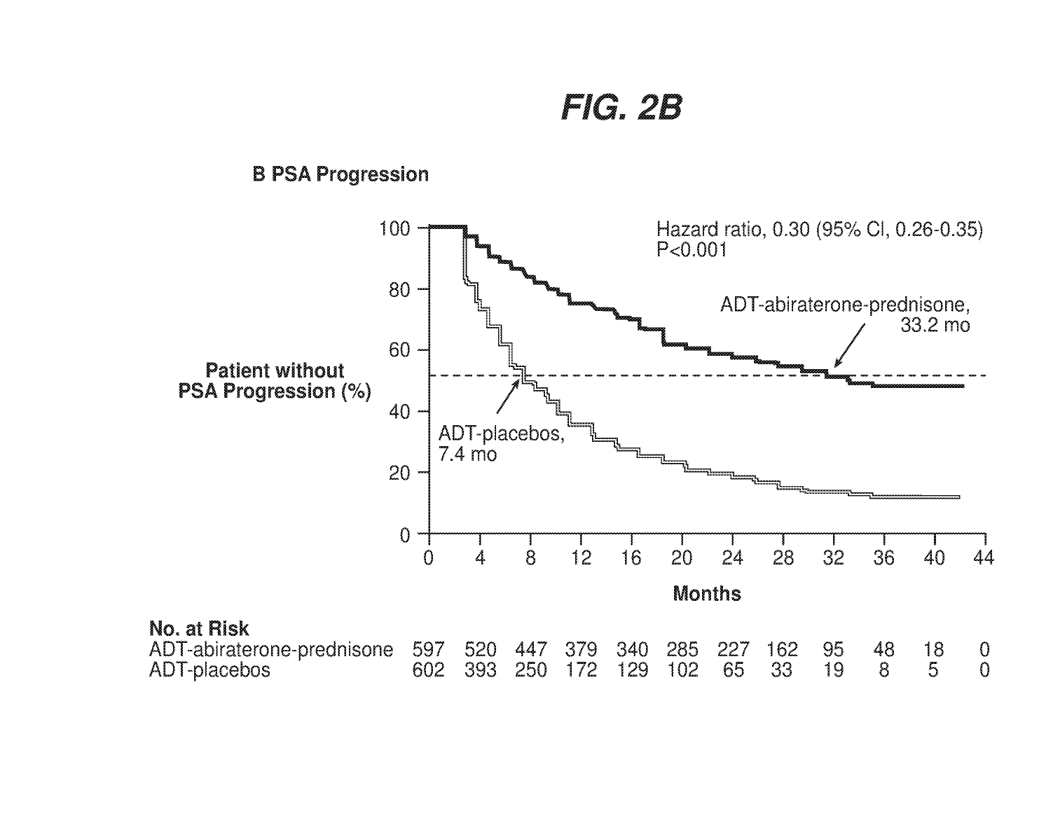

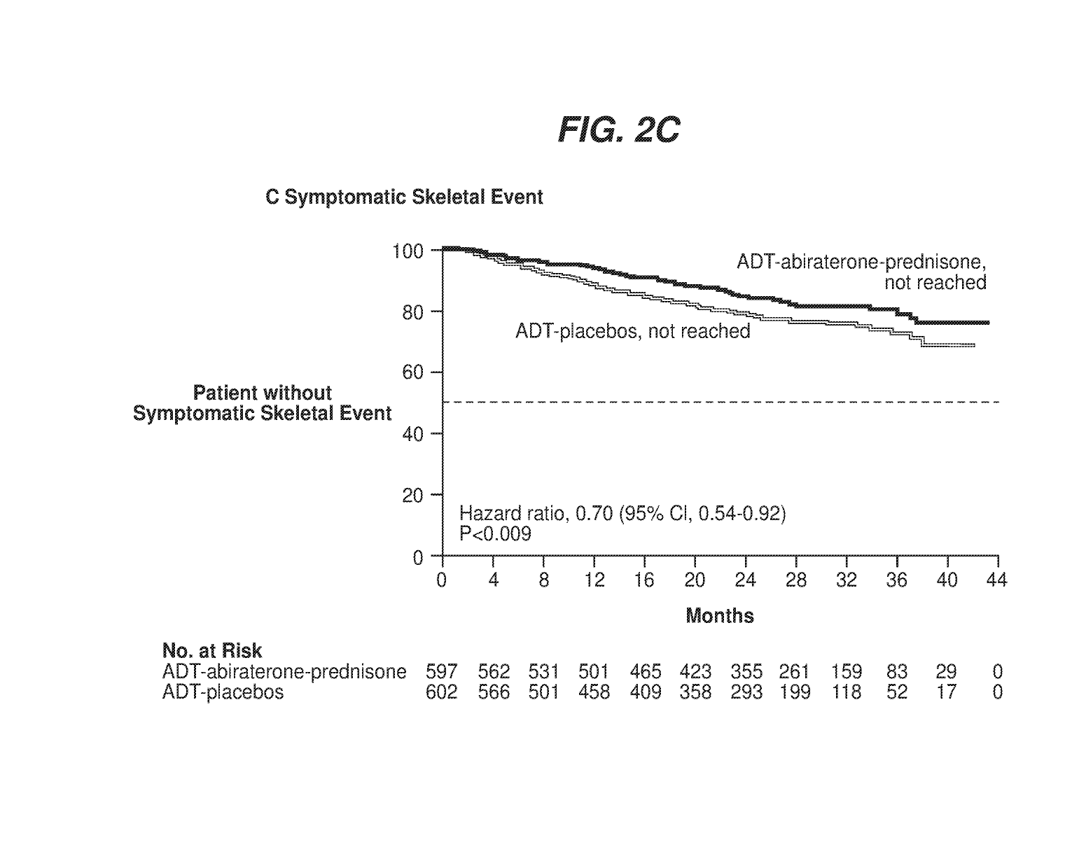

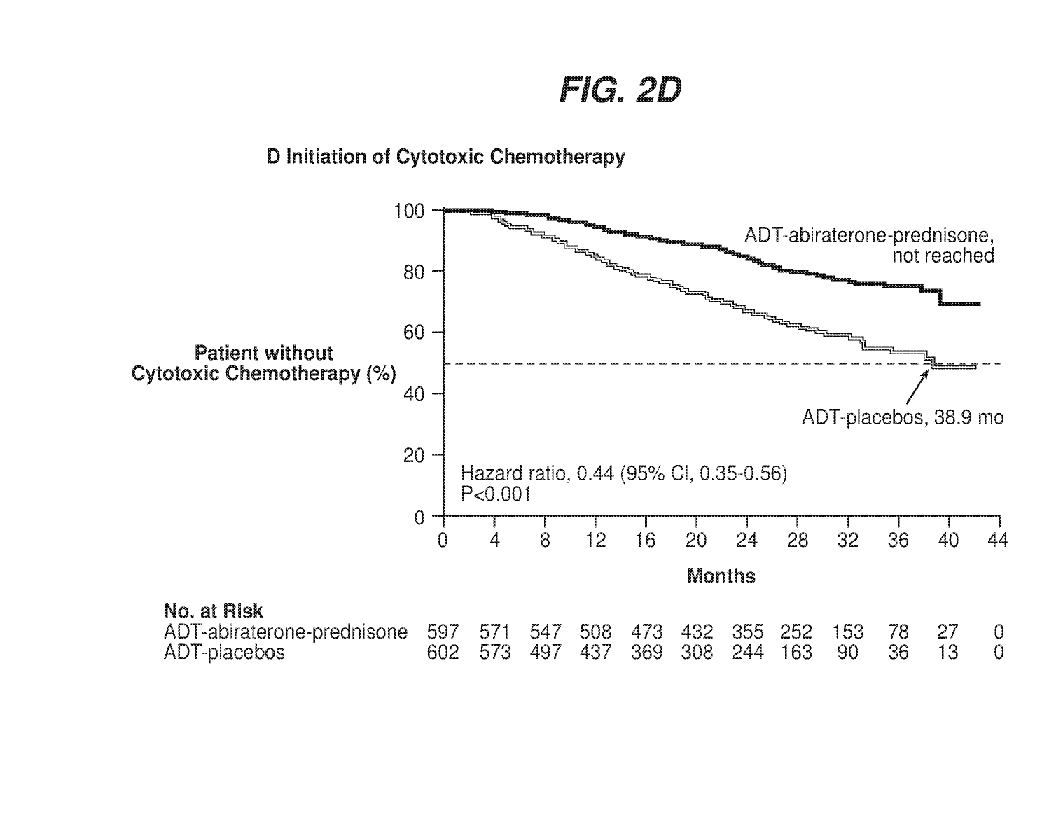

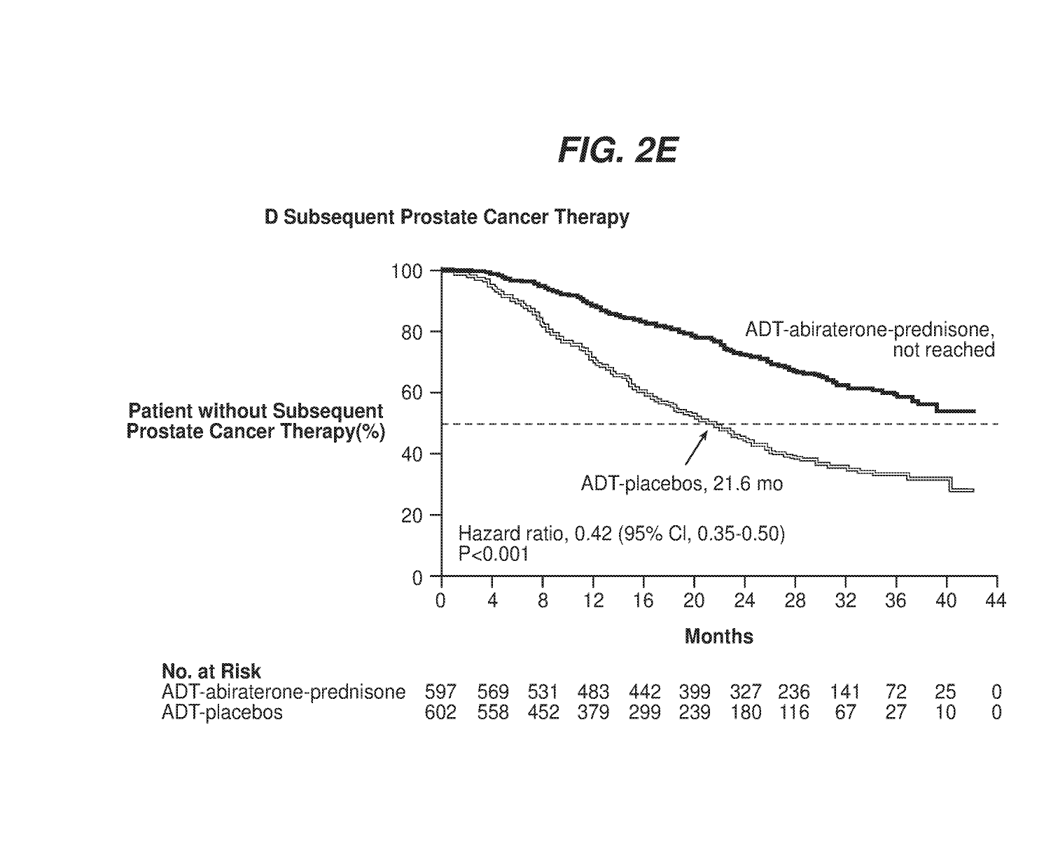

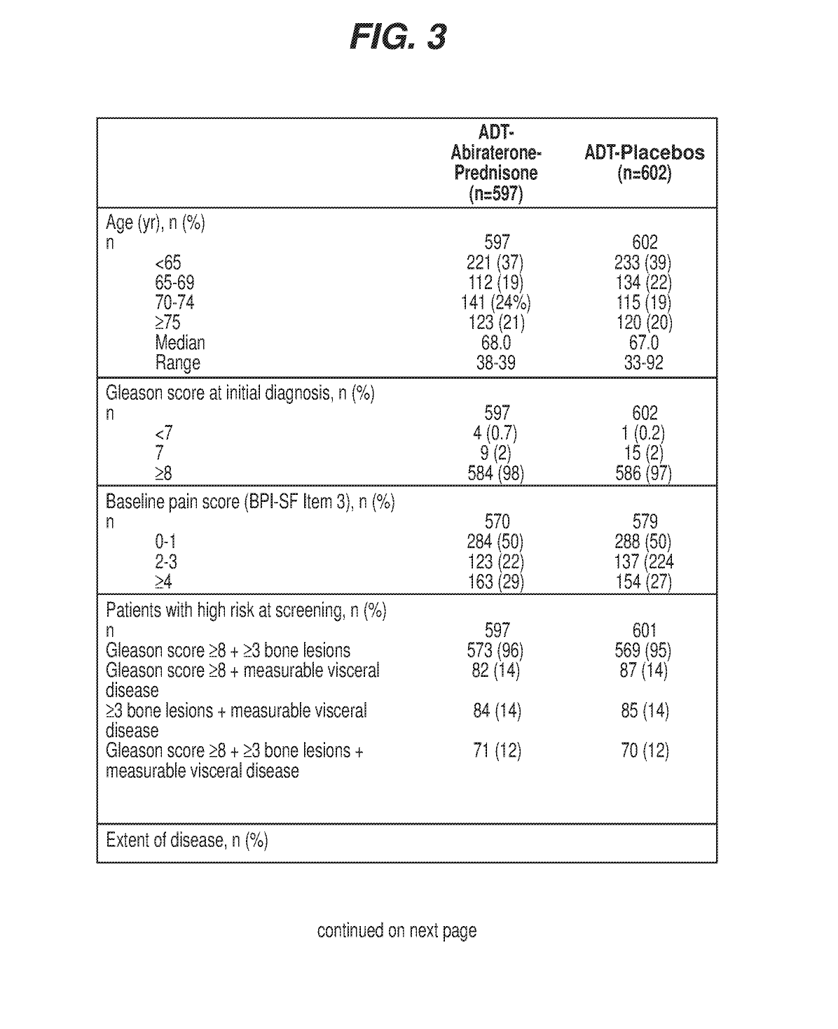

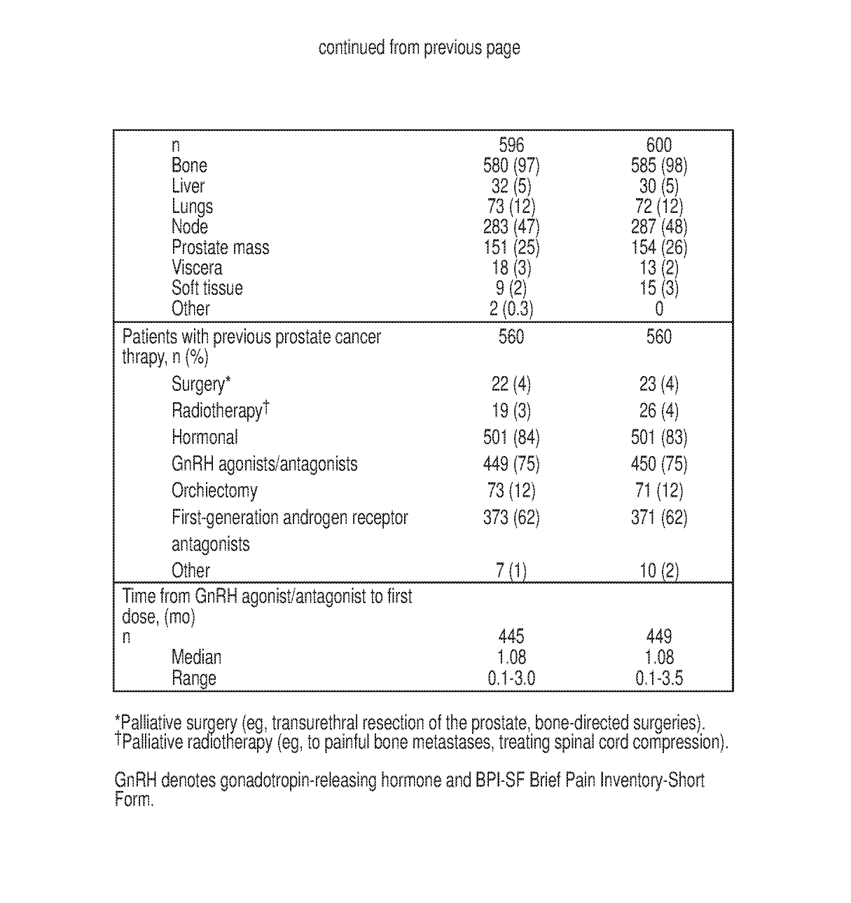

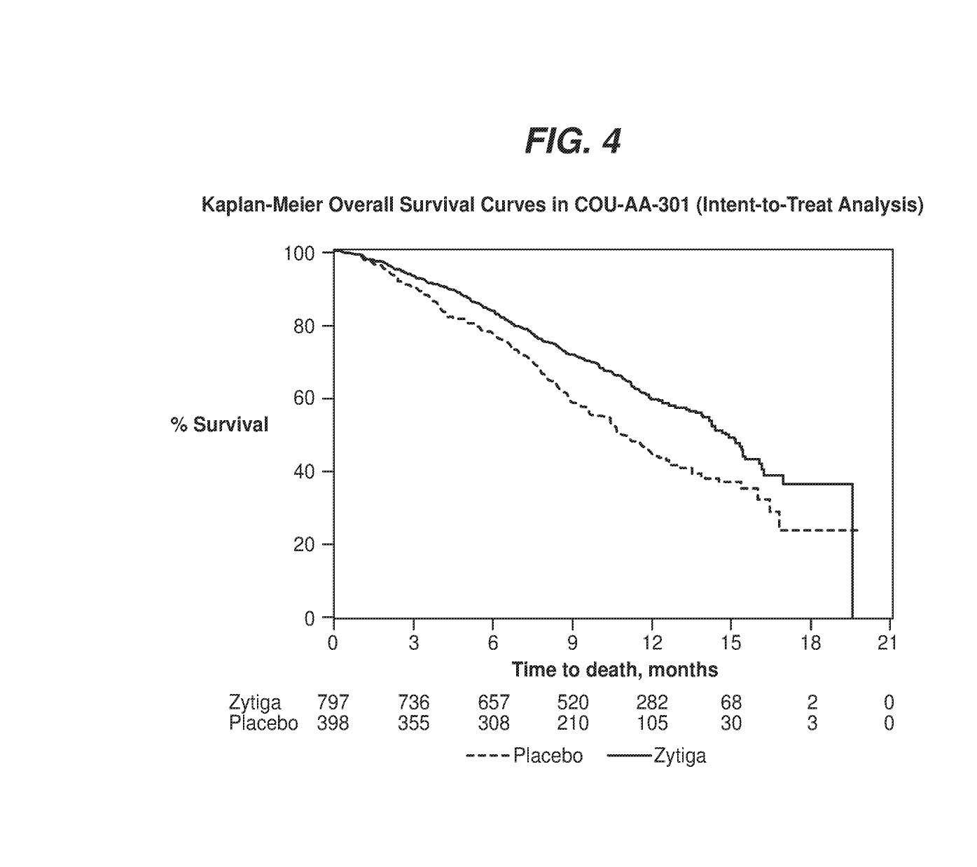

For the LATITUDE study described in Example 1, Kaplan-Meier estimates of overall survival were completed. The first interim analysis was performed, after 406 deaths after a median follow-up duration of 30.4 months; 169 (28%) in the ADT-abiraterone acetate-prednisone group and 237 (39%) in the ADT-placebos group. The median overall survival was not reached for the ADT-abiraterone acetate-prednisone group and 34.7 months (95% CI, 33.1 to not reached) among patients who received ADT-placebos. In reviewing the Kaplan-Meier Estimates of Radiographic Progression-free Survival, Overall Survival, and Subgroup Analyses, the dashed lines indicate the median. The median rate of overall survival was not reached in the ADT-abiraterone-prednisone group and was 34.7 months in the placebo group; the corresponding medians for progression-free survival were 33.0 months and 14.8 months. CI denotes confidence interval. See FIG. 1A.

Men randomized to receive ADT-abiraterone acetate-prednisone had a 38% decrease in the risk of death compared with those receiving ADT-placebos (hazard ratio, 0.62; 95% CI, 0.51 to 0.76, P<0.001).

The results presented are based on the clinical cut-off date of Oct. 31, 2016, for the first interim analysis for overall survival, at which time 48% of 852 deaths had occurred. At a median follow-up of 30.4 months, the median time on treatment was 24 months in the ADT-abiraterone acetate-prednisone group and 14 months in the ADT-placebos group.

The treatment of ADT-abiraterone acetate-prednisone on overall survival was consistently favorable across nearly all pre-specified subgroups. See FIG. 1C.

### Example 5

For the LATITUDE study described in Example 1, Kaplan-Meier estimates of radiographic progression-free survival were completed. At the time of the analysis, treatment with ADT-abiraterone acetate-prednisone resulted in a 53% reduction in the risk of radiographic progression or death compared with ADT-placebos (median 33.0 months vs. median 14.8 months, respectively; hazard ratio, 0.47; 95% CI, 0.39 to 0.55 [P<0.001]). See FIG. 1B.

The results presented are based on the clinical cut-off date of Oct. 31, 2016, for the first interim analysis for overall survival, at which time 593 radiographic progression-free survival events had occurred. At a median follow-up of 30.4 months, the median time on treatment was 24 months in the ADT-abiraterone acetate-prednisone group and 14 months in the ADT-placebos group.

The treatment effect of ADT-abiraterone-prednisone on radiographic progression-free survival was consistent across nearly all pre-specified subgroups. See FIG. 1D.

### Example 6

For the LATITUDE study described in Example 1, secondary endpoints, including pain progression, PSA progression, symptomatic skeletal events, initiation of cytotoxic chemotherapy, and subsequent prostate cancer therapy were assessed. The superiority of ADT-abiraterone acetate-prednisone over ADT-placebos was shown for all secondary end points.

TABLE 2

| | ADT- | | | | |
|---|---|---|---|---|---|
| | Abiraterone- | ADT- | | | |
| | Prednisone | Placebos | Hazard Ratio | | |
| End Point | (n = 597) | (n = 602) | (95% CI) | | P Value^{†} |
| Secondary end points | | | | | |
| Time to pain progression (mo) | NR | 16.6 | 0.70 | (0.58-0.83) | <0.001 |
| Time to PSA progression (mo) | 33.2 | 7.4 | 0.30 | (0.26-0.35) | <0.001 |
| Time to next symptomatic | NR | NR | 0.70 | (0.54-0.92) | 0.009 |
| skeletal event (mo) | | | | | |
| Time to chemotherapy (mo) | NR | 38.9 | 0.44 | (0.35-0.56) | <0.001 |
| Time to subsequent prostate | NR | 21.6 | 0.42 | (0.35-0.50) | <0.001 |
| cancer therapy (mo) | | | | | |
| Exploratory end point | | | | | |
| Patients with a PSA response | 91 | 67 | 1.36 | (1.28-1.45)^{‡} | <0.001 |
| (%) | | | | | |

* CI denotes confidence interval, PSA prostate-specific antigen, and NR not reached.
^{†}P values for secondary end points were calculated by means of a stratified log-rank test and those for the exploratory end point by means of a chi-square test.

‡ A PSA response was defined as a decrease of at least 50% from the baseline value. The comparison for this exploratory end point was calculated as an odds ratio.

### Example 7

For the LATITUDE study described in Example 1, efficacy assessments included sequential radiographic imaging to assess radiographic progression-free survival (CT or MRI and bone scanning) performed every 4 months starting with week 16. PSA concentrations were measured at baseline, monthly in the first year, and then every 2 months until end-of-study treatment. Patients had serial monitoring of vital signs, hematology, serum chemistry, liver function tests, and serum testosterone levels.

Two interim analyses for OS in addition to the final analysis were planned for this study after observing approximately 50% (˜426 death events) and approximately 65% (˜554 death events) of the total number of required (˜852) death events for the final analysis.

Only one analysis for rPFS was performed. The analysis of rPFS was carried out at the two-tailed 0.001 level of significance at the estimated 565 rPFS events. The rPFS analysis occurred in conjunction with the first interim OS analysis. The timing of the first interim analysis was determined according to both rPFS and OS events required, so that the analysis took place when the required number of events in both measures had been reached.

### Example 8

For the LATITUDE study described in Example 1, the Wang-Tsiatis power boundaries with shape parameter 0.2 as implemented by the Lan-DeMets alpha spending method was used for overall survival. Secondary end points were tested using the Hochberg test procedure to control the familywise type I error rate. The primary statistical method of comparison for time-to-event end points was the stratified log-rank test according to the stratification factors. The Cox proportional hazards model was used to estimate the hazard ratio and its associated 95% confidence interval [CI].

### Example 9

For the LATITUDE study described in Example 1, patients receiving one or multiple subsequent therapies totaled 125 (21%) and 246 (41%) in the ADT-abiraterone acetate-prednisone and ADT-placebos groups, respectively. These subsequent therapies were those that have been shown in prospective double-blind randomized phase 3 studies to reduce the risk of death. Docetaxel was the most predominant post-progression treatment in both groups. This analysis was not pre-planned, but rather it was an ad hoc analysis.

TABLE 3

| | | ADT-Abiraterone- | |
|---|---|---|---|
| | | Prednisone | ADT-Placebos |
| | | (n = 597) | (n = 602) |
| | n (%)* | | |
| Agent | | n = 314 | n = 469 |

| | | | | |
|---|---|---|---|---|
| Abiraterone acetate | 10 | (3) | 53 | (11) |
| plus prednisone | | | | |
| Cabazitaxel | 11 | (4) | 30 | (6) |
| Docetaxel | 106 | (34) | 187 | (40) |
| Enzalutamide | 30 | (10) | 76 | (16) |
| Radium-223 | 11 | (4) | 27 | (6) |

### Example 10

For the LATITUDE study described in Example 1, adverse events were recorded. Grade 3 or 4 adverse events were reported in 63% of the patients in the ADT-abiraterone-acetate prednisone group compared with 48% in the ADT-placebos group, as summarized below:

TABLE 4
Summary of Adverse Events

| | ADT-Abiraterone | | | |
|---|---|---|---|---|
| | acetate-Prednisone | | ADT-Placebos | |
| | (n = 597) | | (n = 602) | |
| Adverse Event | number of patients (%) | | | |
| Any adverse event | 558 | (93) | 557 | (93) |
| Grade 3 or 4 adverse event | 374 | (63) | 287 | (48) |
| Any serious adverse event | 165 | (28) | 146 | (24) |
| Any adverse event leading | 73 | (12) | 61 | (10) |
| to treatment discontinuation | | | | |
| Adverse event leading to death | 28 | (5) | 24 | (4) |

The most common, adverse events and events of special interest are listed below. The latter were selected on the basis of the safety profile of phase 2 and phase 3 studies of abiraterone acetate and prednisone.

TABLE 5
Most Common Adverse Events and Events of Special Interest*

| | ADT-Abiraterone Acetate-Prednisone | | | | | | ADT-Placebos | | | | | |
|---|---|---|---|---|---|---|---|---|---|---|---|---|
| | (n = 597) | | | | | | (n = 602) | | | | | |
| | All Grades | | Grade 3 | | Grade 4 | | All Grades | | Grade 3 | | Grade 4 | |
| Adverse Event^{†} | no. of patients (%) | | | | | | | | | | | |
| Hypertension | 219 | (37) | 121 | (20) | 0 | | 133 | (22) | 59 | (10) | 1 | (<1) |
| Hypokalemia | 122 | (20) | 57 | (10) | 5 | (1) | 22 | (4) | 7 | (1) | 1 | (<1) |
| ALT increased | 98 | (16) | 31 | (5) | 2 | (<1) | 77 | (13) | 8 | (1) | 0 | |
| Hyperglycemia | 75 | (13) | 26 | (4) | 1 | (<1) | 68 | (11) | 18 | (3) | 0 | |
| AST increased | 87 | (15) | 25 | (4) | 1 | (<1) | 68 | (11) | 9 | (1) | 0 | |
| Bone pain | 74 | (12) | 20 | (3) | 0 | | 88 | (15) | 17 | (3) | 0 | |
| Cardiac disorder | | | | | | | | | | | | |
| Any | 74 | (12 | 15 | (3 | 5 | (1) | 47 | (8) | 6 | (1) | 0 | |
| Atrial fibrillation | 8 | (1) | 2 | (<1) | <1) | 0 | 2 | (<1) | 1 | (<1) | 0 | |
| Anemia | 54 | (9) | 12 | (2) | 3 | (1) | 85 | (14) | 26 | (4) | 1 | (<1) |

| Back pain | 110 | (18) | 14 | (2) | 0 | 123 | (20) | 19 | (3) | 0 | |
|---|---|---|---|---|---|---|---|---|---|---|---|
| Fatigue | 77 | (13) | 10 | (2) | 0 | 86 | (14) | 14 | (2) | 0 | |
| Spinal cord compression | 14 | (2) | 12 | (2) | 0 | 12 | (2) | 7 | (1) | 3 | (<1) |

^{†}Listed in descending order are events that were reported in at least 2% of the patients in either group. Among other events of special interest, grade 3 peripheral edema was reported in 0.3% of the patients in the ADT-abiraterone acetate-prednisone group and in 0.5% of those in the ADT-placebos group; grade 3 or 4 fluid retention or congestive heart failure was not reported in either group. Grade 3 hot flush was reported in one patient in the ADT-placebos group, and grade 1 irritability was reported in three patients in the ADT-abiraterone acetate-prednisone group.
ALT denotes alanine aminotransferase, and AST aspartate aminotransferase.

The number of patients with serious adverse events was similar between groups. The frequency of adverse events leading to treatment discontinuations was 12% in the ADT-abiraterone-acetate-prednisone group, compared with 10% in the ADT-placebos group, as summarized below:

TABLE 6
Adverse Events Leading to Treatment Discontinuation,
Dose Reduction, and Dose Interruption

| | |
|---|---|
| ADT-Abiraterone- | |
| Prednisone | ADT-Placebos |
| (n = 597) | (n = 602) |

| Adverse Event | no. of patients (%)* |
|---|---|
| | Treatment Discontinuation |

| | | |
|---|---|---|
| All | 73 (12) | 61 (10) |
| Bone pain | 3 (0.5) | 6 (1) |
| Spinal cord compression | 5 (0.8) | 6 (1) |

Dose Reduction

| | | |
|---|---|---|
| All | 55 (9) | 17 (3) |
| ALT increased | 23 (4) | 5 (0.8) |
| AST increased | 12 (2) | 4 (0.7) |
| Hypertension | 8 (1) | 2 (0.3) |
| Hypokalemia | 7 (1) | 0 |

Dose Interruption

| | | |
|---|---|---|
| All | 183(31) | 102 (17) |
| ALT increased | 30 (5) | 10 (2) |
| AST increased | 30 (5) | 9 (2) |
| Hyperglycemia | 6 (1) | 4 (0.7) |
| Hypokalemia | 47 (8) | 4 (0.7) |
| Hypertension | 39 (7) | 15 (2) |
| Nausea | 5 (0.8) | 6 (1) |

*Individual adverse events listed at ≥1% in either group; ALT denotes alanine aminotransferase, and AST aspartate aminotransferase.
A total of 32% of patients in the ADT-abiraterone acetate-prednisone group and 17% in the ADT-placebos group had adverse events that led to dose modification or dose interruption.

### Example 10

### FDA Approved Drug Product Label

THE REMAINDER OF THIS PAGE INTENTIONALLY LEFT BLANK

### Full Prescribing Information

### 1 Indications and Usage

ZYTIGA is indicated in combination with prednisone for the treatment of patients with
Metastatic castration-resistant prostate cancer (CRPC)
Metastatic high-risk castration-sensitive prostate cancer (CSPC)

### 2 Dosage and Administration

### 2.1 Recommended Dose for Metastatic CRPC

The recommended dose of ZYTIGA to 1,000 mg (two 500 mg tablets or four 250 mg tablets) orally once daily with prednisone 5 mg orally twice daily.

### 2.2 Recommended Dose for Metastatic High-Risk CSPC

The recommended dose of ZYTIGA is 1,000 mg (two 500 mg tablets or four 250 mg tablets) orally once daily with prednisone 5 mg administered orally once daily.

### 2.3 Important Administration Instructions

Patients receiving ZYTIGA should also receive a gonadotropin-releasing hormone (GnRH) analog concurrently or should have had bilateral orchiectomy. ZYTIGA must be taken on an empty stomach, either one hour before or two hours after a meal [see Clinical Pharmacology (12.3)]. The tablets should be swallowed whole with water. Do not crush or chew tablets.

### 2.4 Dose Modification Guidelines in Hepatic Impairment and Hepatotoxicity

### Hepatic Impairment

In patients with baseline moderate hepatic impairment (Child-Pugh Class B), reduce the recommended dose of ZYTIGA to 250 mg once daily. In patients with moderate hepatic impairment monitor AST, AST, and bilirubin prior to the start of treatment and monthly thereafter. If elevations in ALT and/or AST greater than 5× upper limit of normal (ULN) or total bilirubin greater than 3×ULN occur in patients with baseline moderate hepatic impairment, discontinue ZYTIGA and do not re-treat patients with ZYTIGA [see Use in Specific Populations (8.6) and Clinical Pharmacology (12.3)].

Do not use ZYTIGA in patients with baseline severe hepatic impairment (Child-Pugh Class C).

### Hepatotoxicity

For patients who develop hepatotoxicity during treatment with ZYTIGA (ALT and/or AST greater than 5×ULN or total bilirubin greater than 3×ULN), interrupt treatment with ZYTIGA [see Warnings and Precautions (5.3)]. Treatment may be restarted at a reduced dose of 750 mg once daily following return of liver function tests to the patient's baseline or to AST and ALT less than or equal to 2.5×ULN and total bilirubin less than or equal to 1.5×ULN. For patients who resume treatment, monitor serum transaminases and bilirubin at a minimum of every two weeks for three months thereafter.

If hepatotoxicity recurs at the dose of 750 mg once daily, re-treatment may be restarted at a reduced dose of 500 mg once daily following return of liver function tests to the patient's baseline or to AST and ALT less than or equal to 2.5×ULN and total bilirubin less than or equal to 1.5×ULN.

If hepatotoxicity recurs at the reduced dose of 300 mg once daily, discontinue treatment with ZYTIGA.

Permanently discontinue ZYTIGA for patients who develop a concurrent elevation of ALT greater than 3×ULN and total bilirubin greater than 2×ULN in the absence of biliary obstruction or other causes responsible for the concurrent elevation [see Warnings and Precautions (5.3)].

### 2.5 Dose Modification Guidelines for Strong CYP 3A4 Inducers

Avoid concomitant strong CYP3A4 inducers (e.g., phenytoin, carbamazepine, refampin, rifabutin, rifapentine, phenobarbital) during ZYTIGA treatment.

If a strong CYP3A4 induces must be co-administered, increase the ZYTIGA dosing frequency to twice a day only during the co-administration period (e.g., from 1,000 mg once daily to 1,000 mg twice a day). Reduce the dose back to the previous dose and frequency, if the concomitant strong CYP3A4 inducer is discontinued [see Drug Interactions (7.1) and Clinical Pharmacology (12.3)].

### 1 Dosage Forms and Strengths

Tablets (500 mg): purple, oval-shaped, film-coated tablets debossed with “AA” one side and “500” on the other side.

Tablets (250 mg): pink, oval-shaped, film-coated tablets debossed with “AA250” on one side.

Tablets (250 mg): white to off-white, oval-shaped tablets debossed with “AA250” on one side.

### 2 Contraindications

### Pregnancy

ZYTIGA can cause fetal harm and potential loss of pregnancy [see Use in Specific Populations (8.1)].

### 1 Warnings and Precautions

### 5.1 Hypertension, Hypokalemia and Fluid Retention Due to Mineralocorticoid Excess

ZYTIGA may cause hypertension, hypokalemia, and fluid retention as a consequence of increased mineralocorticoid levels resulting from CYP17 inhibition [see Clinical Pharmacology (12.1)]. Monitor patients for hypertension, hypokalemia, and fluid retention at least once a month. Control hypertension and correct hypokalemia before and during treatment with ZYTIGA.

In the combined data from 4 placebo-controlled trials using prednisone 5 mg twice daily in combination with 1000 mg abiraterone acetate daily, grades 3-4 hypokalemia were detected in 4% of patients on the ZYTIGA arm and 2% of patients on the placebo arm. Grades 3-4 hypertension were observed 2% of patients each arm and grades 3-4 fluid retention in 1% of patients each arm.

In LATITUDE (a randomized placebo-controlled, multicenter clinical trial), which used prednisone 5 mg daily in combination with 1000 mg abiraterone acetate daily, grades 3-4 hypokalemia were detected in 10% of patients on the ZYTIGA arm and 1% of patients on the placebo arm, grades 3-4 hypertension were observed in 20% of patients on the ZYTIGA arm and 10% of patients on the placebo arm. Grades 3-4 fluid retention occurred in 1% of patients each arm [see Adverse Reactions (6)].

Closely monitor patients whose underlying medical conditions might be compromised by increases in blood pressure, hypokalemia or fluid retention, such as those with heart failure, recent myocardial infarction, cardiovascular disease, or ventricular arrhythmia. The safety of ZYTIGA in patients with left ventricular ejection fraction <50% or New York Heart Association (NYHA) Class III or IV heart failure (in COU-AA-301) or NYHA Class II to IV heart failure (in COU-AA-302 and LATITUDE) has not been established because there patients were excluded from these randomized clinical trials [see Clinical Studies (14)].

### 5.2 Adrenocortical Insufficiency

Adrenal insufficiency occurred in 0.3% of 2230 patients taking ZYTIGA and in 0.1% of 1763 patients taking placebo in the combined data of the 5 randomized, placebo-controlled clinical studies. Adrenocortical insufficiency was reported in patients receiving ZYTIGA in combination with prednisone, following interruption of daily steroids and/or with concurrent infection or stress.

Monitor patients for symptoms and signs of adrenocortical insufficiency, particularly if patients are withdrawn from prednisone, have prednisone dose seductions, or experience unusual stress. Symptoms and signs of adrenocortical insufficiency may be masked by adverse reactions associated with mineralocorticoid excess seen so patients treated with ZYTIGA. If clinically indicated, perform appropriate tests to confirm the diagnosis of the adrenocortical insufficiency. Increased dosage of corticosteroids may be indicated before, during and after stressful situations [see Warnings and Precautions (5.1)].

### 5.3 Hepatotoxicity

In postmarketing experience, there have been ZYTIGA-associated severe hepatic toxicity, including fulminant hepatitis, acute liver failure and deaths [see Adverse Reactions (6.2)].

In the combined data of 5 randomized clinical trials, grade 3-4 ALT or AST increases (at least 5×ULN) were reported 6% of 2230 patients who received ZYTIGA, typically during the first 3 months after starting treatment. Patients whose baseline ALT or AST were elevated were more likely to experience liver test elevation than those beginning with normal values. Treatment discontinuation due to ALT and AST increases or abnormal hepatic function occurred in 1.1% of 2230 patients taking ZYTIGA. In these clinical trials, no deaths clearly related to ZYTIGA were reported due to hepatotoxicity events.

Measure serum transaminases (ALT and AST) and bilirubin levels prior to starting treatment with ZYTIGA, every two weeks for the first three months of treatment and monthly thereafter. In patients with baseline moderate hepatic impairment receiving a reduced ZYTIGA dose of 250 mg, measure ALT, AST, and bilirubin prior so the start of treatment, every week for the first month, every two weeks for the following two months of treatment and monthly thereafter. Promptly measure serum total bilirubin, AST, and ALT if clinical symptoms or signs suggestive of hepatotoxicity develop. Elevations of AST, ALT, or bilirubin from the patient's baseline should prompt more frequent monitoring. If at any time AST or ALT rise above five times the ULN, or the bilirubin rises above three times the ULN, interrupt ZYTIGA treatment and closely monitor liver function.

Re-treatment with ZYTIGA at a reduced dose level may take place only after return of liver function tests to the patient's baseline or to AST and ALT less than or equal to 2.5×ULN and total bilirubin less than or equal to 1.5×ULN [see Dosage and Administration (2.5)].

Permanently discontinue ZYTIGA for patients who develop a concurrent elevation of ALT greater than 3×ULN and total bilirubin greater than 2×LN in the absence of biliary obstruction or other causes responsible for the concurrent elevation [see Dosage and Administration (2.4)].

The safety of ZYTIGA re-treatment of patients who develop AST or ALT greater than or equal to 20×ULN and/or bilirubin greater than or equal to 10×ULN is unknown.

### 1 Adverse Reactions

The following are discussed in more detail in other sections of the labeling:
Hypertension, Hypokalemia, and Fluid Retention due to Mineralocorticoid Excess [see Warnings and Precaution (5.1)].
Adrenocortical Insufficiency [see Warnings and Precautions (5.2)].
Hepatotoxicity [see Warnings and Precautions (5.3)].

### 6.1 Clinical Trial Experience

Because clinical trials are conducted under widely varying conditions, adverse reaction rates observed in the clinical trials of a drag cannot be directly compared to rates in the clinical trials of another drug and may not reflect the rates observed in clinical practice.

Two randomized placebo-controlled, multicenter clinical trials (COU-AA-301 and COU-AA-302) enrolled patients who had metastatic CRPC in which ZYTIGA was administered orally at a dose of 1,000 mg daily in combination with prednisone 5 mg twice daily in the active treatment arms. Placebo plus prednisone 5 mg twice daily was given to patients on the control arm. A third randomized placebo-controlled, multicenter clinical trial (LATITUDE) enrolled patients who had metastatic high-risk CSPC in which ZYTIGA was administered at a dose of 1,000 mg daily in combination with prednisone 5 mg once daily. Placebos were administered to patients in the control arm. Additionally, two other randomized, placebo-controlled, trials were conducted in patients with metastatic CRPC. The safety data pooled from 2230 patients in the 5 randomized controlled trials constitute the basis for the data presented in the Warnings and Precautions, Grade 1-4 adverse reactions, and Grade 1-4 laboratory abnormalities. In all trials, a gonadotropin-releasing hormone (GnRH) analog or prior orchiectomy was required in both arms.

In the pooled data, median treatment duration was 11 months (0.1, 43) for ZYTIGA-treated patients and 7.2 months (0.1, 43) for placebo-treated patients. The most common adverse reactions (≥10%) that occurred more commonly (>2%) in the ZYTIGA arm were fatigue, arthralgia, hypertension, nausea, edema, hypokalemia, hot flush, diarrhea, vomiting, upper respiratory infection, cough, and headache. The most common laboratory abnormalities (>20%) that occurred more commonly (≥2%) in the ZYTIGA arm were anemia, elevated alkaline phosphatase, hypertriglyceridemia, lymphopenia, hypercholesterolemia, hyperglycemia, and hypokalemia. Grades 3-4 adverse events were reported for 53% of patients in the ZYTIGA arm and 46% of patients hi the placebo arm. Treatment discontinuation was reported in 14% of patients in the ZYTIGA arm and 13% of patients in the placebo arm. The common adverse events (≥1%) resulting in discontinuation of ZYTIGA and prednisone were hepatotoxicity and cardiac disorders.

Deaths associated with treatment-emergent adverse events were reported for 7.5% of patients in the ZYTIGA arm and 6.6% of patients in the placebo arm. Of the patients in the ZYTIGA arm, the most common cause of death was disease progressions (3.3%). Other reported causes of death in ≥5 patients included pneumonia, cardio-respiratory arrest, death (no additional information), and general physical health deterioration.

### COU-AA-301: Metastatic CRPC Following Chemotherapy

COU-AA-301 enrolled 1195 patients with metastatic CRPC who had received prior docetaxel chemotherapy. Patients were not eligible if AST and/or ALT ≥2.5×LN in the absence of liver metastases. Patients with liver metastases were excluded if AST and/or ALT≥5×ULN.

Table 7 shows adverse reactions on the ZYTIGA are in COU-AA-301 that occurred with a ≥2% absolute increase in frequency compared to placebo or were events of special interest. The median duration of treatment with ZYTIGA with prednisone was 8 months.

TABLE 7
Adverse Reactions due to ZYTIGA in COU-AA-301

| | ZYTIGA with | | Placebo with | |
|---|---|---|---|---|
| | Prednisone | | Prednisone | |
| | (N = 791) | | (N = 394) | |
| | All | Grade | All | Grade |
| System/Organ Class | Grades¹ | 3-4 | Grades | 3-4 |
| Adverse reaction | % | % | % | % |
| Musculoskeletal and | | | | |
| connective tissue disorders | | | | |
| Joint swelling/discomfort² | 30 | 4.2 | 23 | 4.1 |
| Muscle discomfort³ | 26 | 3.0 | 23 | 2.3 |
| General disorders | | | | |
| Edema⁴ | 27 | 1.9 | 18 | 0.8 |
| Vascular disorders | | | | |
| Hot flush | 19 | 0.3 | 17 | 0.3 |
| Hypertension | 8.5 | 1.3 | 6.9 | 0.3 |
| Gastrointestinal disorders | | | | |
| Diarrhea | 18 | 0.6 | 14 | 1.3 |
| Dyspepsia | 6.1 | 0 | 3.3 | 0 |
| Infections and infestations | | | | |
| Urinary tract infection | 12 | 2.1 | 7.1 | 0.5 |
| Upper respiratory tract infection | 5.4 | 0 | 2.5 | 0 |
| Respiratory, thoracic and | | | | |
| mediastinal disorders | | | | |
| Cough | 11 | 0 | 7.6 | 0 |
| Renal and urinary disorders | | | | |
| Urinary frequency | 7.2 | 0.3 | 5.1 | 0.3 |
| Nocturia | 6.2 | 0 | 4.1 | 0 |
| Injury, poisoning and | | | | |
| procedural complications | | | | |
| Fractures⁵ | 5.9 | 1.4 | 2.3 | 0 |
| Cardiac disorders | | | | |
| Arrhythmia⁶ | 7.2 | 1.1 | 4.6 | 1.0 |
| Chest pain or chest discomfort⁷ | 3.8 | 0.5 | 2.8 | 0 |
| Cardiac failure⁸ | 2.3 | 1.9 | 1.0 | 0.3 |

¹Adverse events graded according to CTCAE version 3.0.
²Includes terms Arthritis, Arthralgia, Joint swelling, and Joint stiffness.
³Includes terms Muscle spasms, Musculoskeletal pain, Myalgia, Musculoskeletal discomfort, and Musculoskeletal stiffiness.
⁴Includes terms Edema, Edema peripheral, Pitting edema, and Generalized edema.
⁵Includes all fractures with the exception of pathological fracture.
⁶Includes terms Arrhythmia, Tachycardia, Atrial fibrillation, Supraventricular tachycardia, Atrial tachycardia, Ventricular tachycardia, Atrial flutter, Bradycardia, Atrioventricular block complete. Conduction disorder, and Bradyarrhythmia.
⁷Includes terms Angina pectoris, Chest pain, and Angina unstable. Myocardial infarction or ischemia occured more commonly in the placebo arm than in the ZYTIGA arm (1.3% vs. 1.1% respectively).
⁸Includes terms Cardiac failure, Cardiac failure congestive, Left ventricular dysfunction. Cardiogenic shock, Cardiomegaly, Cardiomyopathy, and Ejection fraction decreased.

Table 8 shows laboratory abnormalities of interest from COU-AA-301.

TABLE 8
Laboratory Abnormalities of Interest in COU-AA-301

| | ZYTIGA with | | Placebo with | |
|---|---|---|---|---|
| | Prednisone | | Prednisone | |
| | (N = 791) | | (N = 394) | |
| | All | Grade | All | Grade |
| Laboratory | Grades | 3-4 | Grades | 3-4 |
| Abnormality | (%) | (%) | (%) | (%) |
| Hypertriglyceridemia | 63 | 0.4 | 53 | 0 |
| High AST | 31 | 2.1 | 36 | 1.5 |
| Hypokalemia | 28 | 5.3 | 20 | 1.0 |
| Hypophosphatemin | 24 | 7.2 | 16 | 5.8 |
| High ALT | 11 | 1.4 | 10 | 0.8 |
| High Total Bilirubin | 6.6 | 0.1 | 4.6 | 0 |

### COU-AA-302: Metastatic CRPC Prior to Chemotherapy

COU-AA-302 enrolled 1088 patients with metastatic CRPC who had not received prior cytotoxic chemotherapy. Patients were ineligible if AST and/or ALT≥2.5×ULN and patients were excluded if they had liver metastases.

Table 9 shows adverse reactions on the ZYTIGA are in COU-AA-302 that occurred in ≥5% of patients with a ≥2% absolute increase in frequency compared to placebo. The median duration of treatment with ZYTIGA with prednisone was 13.8 months.

TABLE 9
Adverse Reactions in ≥5% of Patients
on the ZYTIGA Arm in COU-AA-302

| | ZYTIGA with | | Placebo with | |
|---|---|---|---|---|
| | Prednisone | | Prednisone | |
| | (N = 542) | | (N = 540) | |
| | All | Grade | All | Grade |
| System/Organ Class | Grades¹ | 3-4 | Grades | 3-4 |
| Adverse reaction | % | % | % | % |
| General disorders | | | | |
| Fatigue | 39 | 2.2 | 34 | 1.7 |
| Edema² | 25 | 0.4 | 21 | 1.1 |
| Pyrexia | 8.7 | 0.6 | 5.9 | 0.2 |
| Musculoskeletal and | | | | |
| connective tissue disorders | | | | |
| Joint swelling/discomfort³ | 30 | 2.0 | 25 | 2.0 |
| Groin pain | 6.6 | 0.4 | 4.1 | 0.7 |
| Gastrointestinal disorders | | | | |
| Constipation | 23 | 0.4 | 19 | 0.6 |
| Diarrhea | 22 | 0.9 | 18 | 0.9 |
| Dyspepsia | 11 | 0.0 | 5.0 | 0.2 |
| Vascular disorders | | | | |
| Hot flush | 22 | 0.2 | 18 | 0.0 |
| Hypertension | 22 | 3.9 | 13 | 3.0 |
| Respiratory, thoracic and | | | | |
| mediastimal disorders | | | | |
| Cough | 17 | 0.0 | 14 | 0.2 |
| Dyspnea | 12 | 2.4 | 9.6 | 0.9 |
| Psychiatric disorders | | | | |
| Insomnia | 14 | 0.2 | 11 | 0.0 |
| Injury, poisoning and | | | | |
| procedural complications | | | | |
| Contusion | 13 | 0.0 | 9.1 | 0.0 |
| Falls | 5.9 | 0.0 | 3.3 | 0.0 |
| Infections and infestations | | | | |
| Upper respiratory tract infection | 13 | 0.0 | 8.0 | 0.0 |
| Nasopharyngitis | 11 | 0.0 | 8.1 | 0.0 |
| Renal and urinary disorders | | | | |
| Hematuria | 10 | 1.3 | 5.6 | 0.6 |
| Skin and subcutaneous | | | | |
| tissue disorders | | | | |
| Rash | 8.1 | 0.0 | 3.7 | 0.0 |

¹Adverse events graded according to CTCAE version 3.0.
²Includes terms Edema peripheral, Pitting edema, and Generalized edema.
³Includes terms Arthritis, Arthralgia, Joint swelling, and Joint stiffness.

Table 10 shows laboratory abnormalities that occurred in greater than 15% of patients, and more frequently (>5%) in the ZYTIGA arm compared to placebo in COU-AA-302.

TABLE 10
Laboratory Abnormalities in >15% of
Patients in the ZYTIGA Arm of COU-AA-302

| | |
|---|---|
| ZYTIGA with | Placebo with |
| Prednisone | Prednisone |
| (N = 542) | (N = 540) |

| | Grade | Grade | Grade | Grade |
|---|---|---|---|---|
| Laboratory | 1-4 | 3-4 | 1-4 | 3-4 |
| Abnormality | % | % | % | % |
| Hematology | | | | |
| Lymphopenia | 38 | 8.7 | 32 | 7.4 |
| Chemistry | | | | |
| Hyperglycemia¹ | 57 | 6.5 | 51 | 5.2 |
| High ALT | 42 | 6.1 | 29 | 0.7 |
| High AST | 37 | 3.1 | 29 | 1.1 |
| Hypernatremia | 33 | 0.4 | 25 | 0.2 |
| Hypokalemia | 17 | 2.8 | 10 | 1.7 |

¹Based on non-fasting blood draws

LATITUDE: Patients with Metastatic High-Risk CSPC

LATITUDE enrolled 1199 patients with newly-diagnosed metastatic, high-risk CSPC who had not received prior cytotoxic chemotherapy. Patients were ineligible if AST and/or ALT≥2.5×ULN or if they had liver metastases. All the patients received GnRH analogs or had prior bilateral orchiectomy during the trial. The median duration of treatment with ZYTIGA and prednisone was 24 months.

Table 11 shows adverse reactions on the ZYTIGA arm that occurred in ≥5% of patients with a ≥2% absolute increase in frequency compared to those on the placebos arm.

TABLE 11
Adverse Reactions in ≥5% of Patients
of the ZYTIGA Arm in LATITUDE¹

| | ZYTIGA with | | | |
|---|---|---|---|---|
| | Prednisone | | Placebos | |
| | (N = 597) | | (N = 602) | |
| | All | Grade | All | Grades |
| System/Organ Class | Grades² | 3-4 | Grades | 3-4 |
| Adverse reaction | % | % | % | % |
| Vascular disorders | | | | |
| Hypertension | 37 | 20 | 13 | 10 |
| Hot flush | 15 | 0.0 | 13 | 0.2 |
| Metabolism and | | | | |
| nutrition disorders | | | | |
| Hypokalemia | 20 | 10 | 3.7 | 1.3 |
| Investigations | | | | |
| Alanine aminotransferase | 16 | 5.5 | 13 | 1.3 |
| increased³ | | | | |
| Aspartate aminotransferase | 15 | 4.4 | 11 | 1.5 |
| increased³ | | | | |
| Infections and infestations | | | | |
| Urinary tract infections | 7.0 | 1.0 | 3.7 | 0.8 |
| Upper respiratory tract infection | 6.7 | 0.2 | 4.7 | 0.2 |
| Nervous system disorders | | | | |
| Headache | 7.5 | 0.3 | 5.0 | 0.2 |
| Respiratory, Thoracic and | | | | |
| Mediastinal Disorders | | | | |
| Cough⁴ | 6.5 | 0.0 | 3.2 | 0 |

¹All patients were receiving an GnRH agonist or had undergone orchiectomy.
²Adverse events graded according to CTCAE version 4.0
³Reported as an adverse event or reaction
⁴Including cough, productive cough, upper airway cough syndrome

Table 12 shows laboratory abnormalities that occurred in >15% of patients, and more frequently (>5%) in the ZYTIGA arm compared to placebos.

TABLE 12
Laboratory Abnormalities in >15% of
Patients in the ZYTIGA Arm of LATITUDE

| | ZYTIGA with | | | |
|---|---|---|---|---|
| | Prednisone | | Placebos | |
| | (N = 597) | | (N = 602) | |
| | Grade | Grade | Grade | Grade |
| Laboratory | 1-4 | 3-4 | 1-4 | 3-4 |
| Abnormality | % | % | % | % |
| Hematology | | | | |
| Lymphopenia | 20 | 4.1 | 14 | 1.8 |
| Chemistry | | | | |
| Hypokalemia | 30 | 9.6 | 6.7 | 1.3 |
| Elevated ALT | 46 | 6.4 | 45 | 1.3 |
| Elevated total bilirubin | 16 | 0.2 | 6.2 | 0.2 |

### Cardiovascular Adverse Reactions

In the combined data of 5 randomized, placebo-controlled clinical studies, cardiac failure occurred more commonly in patients on the ZYTIGA arm compared to patients on the placebo arm (2.6% versus 0.9%). Grade 3-4 cardiac failure occurred in 1.3% of patients taking ZYTIGA and led to 5 treatment discontinuations and 4 deaths. Grade 3-4 cardiac failure occurred in 0.2% of patients taking placebo. There were no treatment discontinuations and two deaths due to cardiac failure in the placebo group.

In the same combined data, the majority of arrhythmias were grade 1 or 2. There was one death associated with arrhythmia and three patients with sudden death in the ZYTIGA arms and five deaths in the placebo arms. These were 7 (0.3%) deaths due to cardiorespiratory arrest in the ZYTIGA arms and 2 (0.1%) deaths in the placebo arms. Myocardial ischemia or myocardial infection led to death in 3 patients in the placebo arms and 3 deaths in the ZYTIGA arms.

### 6.2 Postmarketing Experience

The following additional adverse reactions have been identified during post approval use of ZYTIGA with prednisone. Because these reactions are reported voluntarily from a population of uncertain size, it is not always possible to reliably estimate their frequency or establish a casual relationship to drug exposure.

Respiratory, Thoracic and Mediastinal Disorders: non-infectious pneumonitis.

Musculoskeletal and Connective Tissue Disorders: myopathy, including rhabdomyolysis.

Hepatobiliary Disorders: fulminant hepatitis, including acute hepatic failure and death.

### 7 Drug Interactions

7.1 Drugs that Inhibit or Induce CYP3A4 Enzymes

Based on in vitro data, ZYTIGA is a substrate of CYP3A4.

In a dedicated drug interaction trial, co-administration of rifampin, a strong CYP3A4 inducer, decreased exposure of abiraterone by 55%. Avoid concomitant strong CYP3A4 inducers during ZYTIGA treatment If a strong CYP3A4 inducer must be co-administered, increase the ZYTIGA dosing frequency [see Dosage and Administration (2.5) and Clinical Pharmacology (12.3)].

In a dedicated drug interaction trial, co-administration of ketoconazole, a strong inhibitor of CYP3A4, had no clinically meaningful effect on the pharmacokinetics of abiraterone [see Clinical Pharmacology (12.3)].

### 7.2 Effects of Abiraterone on Drug Metabolizing Enzymes

ZYTIGA is an inhibitor of the hepatic drug-metabolizing enzymes CYP2D6 and CYP2D8. In a CYP2D6 drug-drug interaction trial, the Cₘₐₓ and AUC of dextromethorphan (CYP2D6 substrate) were increased 2.8- and 2.9-fold, respectively, when dextromethorphan was given with abiraterone acetate 1,000 mg daily and prednisone 5 mg twice daily. Avoid co-administration of abiraterone acetate with substrates of CYP2D6 with a narrow therapeutic index (e.g., thioridazine). If alternative treatments cannot be used, consider a dose reduction of the concomitant CYP2D6 substrate drug [see Clinical Pharmacology (12.3)].

In a CYP2C8 drug-drug interaction trial in healthy subjects, the AUC of pioglitazone (CYP2C8 substrate) was increased by 46% when pioglitazone was given together with a single dose of 1,000 mg abiraterone acetate. Therefore, patients should be monitored closely for signs of toxicity related to a CYP2C8 substrate with a narrow therapeutic index if used concomitantly with ZYTIGA [see Clinical Pharmacology (12.3)].

### 8 Use in Specific Populations

### 8.1 Pregnancy

### Risk Summary

Based on findings from animal studies and the mechanism of action, ZYTIGA is contraindicated for use in pregnant women because the drug can cause fetal harm and potential loss of pregnancy. ZYTIGA is not indicated for use in females.

### Animal Data

In an embryo-fetal developmental toxicity study in rats, abiraterone acetate caused developmental toxicity when administered at oral doses of ID, 30 or 100 mg/kg/day throughout the period of organogenesis (gestational days 6-17). Findings included embryo-fetal lethality (increased post implantation loss and resorptions and decreased number of live fetuses), fetal developmental delay (skeletal effects) and urogenital effects (bilateral ureter dilation) at doses ≥10 mg/kg/day, decreased fetal ano-genital distance at ≥30 mg/kg/day, and decreased fetal body weight at 100 mg/kg/day. Doses ≥10 mg/kg/day caused maternal toxicity. The doses tested in rats resulted in systemic exposures (AUC) approximately 0.03,0.1 and 0.3 times, respectively, the AUC in patients.

### Lactation

### Risk Summary

ZYTIGA is not indicated for use in women. There is no information available on the presence of abiraterone acetate in human milk, or on the effects on the breastfed child or milk production.

### Females and Males of Reproductive Potential

### Risk Summary

### Males

Based on findings in animal reproduction studies and its mechanism of action, ad vise males with female partners of reproductive potential to use effective contraception during treatment and for 3 weeks after the final dose of ZYTIGA [see Use in Specific Populations (8.1)].

### Infertility

Based on animal studies, ZYTIGA may impair reproductive function and fertility in males of reproductive potential [see Nonclinical Toxicology (13.1)].

### 8.4 Pediatric Use

Safety and effectiveness of ZYTIGA in pediatric patients have not been established.

### 8.5 Geriatric Use

Of the total number of patients receiving ZYTIGA in randomized clinical trials 70% of patients were 65 years and over and 21% were 75 years and over. No overall differences in safety or effectiveness were observed between these elderly patients and younger patients. Other reported clinical experience has not identified differences in responses between the elderly and younger patients, but greater sensitivity of some older individuals cannot be ruled out.

8.6 Patients with Hepatic Impairment

The pharmacokinetics of abiraterone were examined in subjects with baseline mild (N=8) or moderate (N=8) hepatic impairment (Child-Pugh Class A and B, respectively) and in 8 healthy control subjects with normal hepatic function. The systemic exposure (AUC) of abiraterone after a single oral 1,000 mg dose of ZYTIGA increased by approximately 1,1-fold and 3.6-fold in subjects with mild and moderate baseline hepatic impairment, respectively compared to subjects with normal hepatic function.

In another trial, the pharmacokinetics of abiraterone were examined in subjects with baseline severe (N=8) hepatic impairment (Child-Pugh Class C) and in 8 healthy control subjects with normal hepatic function. The systemic exposure (AUC) of abiraterone increased by approximately 7-fold and the fraction of free drug increased 2-fold in subjects with severe baseline hepatic impairment compared to subjects with normal hepatic function.

No dosage adjustment is necessary for patients with baseline mild hepatic impairment. In patients with baseline moderate hepatic impairment (Child-Pugh Class B), reduce the recommended dose of ZYTIGA to 250 mg once daily. Do not use ZYTIGA in patients with baseline severe hepatic impairment (Child-Pugh Class C). If elevations in ALT or AST>5×ULN or total bilirubin >3×ULN occur in patients with baseline moderate hepatic impairment, discontinue ZYTIGA treatment [see Dosage and Administration (2.4) and Clinical Pharmacology (12.3)].

For patients who develop hepatotoxicity during treatment interruption of treatment and dosage adjustment may be required [see Dosage and Administration (2.4), Warnings and Precautions (5.3), and Clinical Pharmacology (12.3)].

8.7 Patients with Renal Impairment

No dosage adjustment is necessary for patients with renal impairment [see Clinical Pharmacology (12.3)].

### 10 Overdosage

Human experience of overdose with ZYTIGA is limited.

There is no specific antidote. In the event of an overdose, stop ZYTIGA, undertake general supportive measures, including monitoring for arrhythmias and cardiac failure and assess liver function.

### DESCRIPTION

Abiraterone acetate, the active ingredient of ZYTIGA is the acetyl ester of abiraterone. Abiraterone is an inhibitor of CYP17 (17α-hydroxylase/C17,20-lyase). Each ZYTIGA tablet contains either 250 mg or 500 mg of abiraterone acetate. Abiraterone acetate is designated chemically as (3β)-17-(3-pyridinyl) androsta-5,16-dien-3-yl acetate and its structure is:

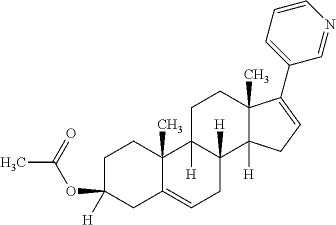

Abiraterone acetate is a white to off-white, non-hygroscopic, crystalline powder. Its molecular formula is C₂₆H₃₃NO₂ and it has a molecular weight of 391.55. Abiraterone acetate is a lipophilic compound with an octanol-water partition coefficient of 5.12 (Log P) and is practically insoluble in water. The pKa of the aromatic nitrogen is 5.19.

ZYTIGA tablets are available in 500 mg film-coated tablets, 250 mg film-coated tablets and 250 mg uncoated tablets with the following inactive ingredients:
500 mg film-coated tablets: colloidal silicon dioxide, croscarmellose sodium, hypromellose, lactose monohydrate, magnesium stearate, silicified microcrystalline cellulose, and sodium lauryl sulfate. The coating, Opadry® II Purple, contains iron oxide black, iron oxide red, polyethylene glycol, polyvinyl alcohol, talc, and titanium dioxide.
250 mg film-coated tablets: colloidal silicon dioxide, croscarmellose sodium, lactose monohydrate magnesium stearate microcrystalline cellulose, povidone, and sodium lauryl sulfate. The coating, Opadry® II Beige, comas as iron oxide red, iron oxide yellow, polyethylene glycol, polyvinyl alcohol, talc, and titanium dioxide.
250 mg uncoated tablets: colloidal silicon dioxide, croscarmellose sodium, lactose monohydrate, magnesium stearate, microcrystalline cellulose, povidone, and sodium lauryl sulfate.

### Clinical Pharmacology

### 12.1 Mechanism of Action

Abiraterone acetate (ZYTIGA) is converted in vivo to abiraterone, an androgen biosynthesis inhibitor, that inhibits 17α-hydroxylase/C17,20-lyase (CYP17). This enzyme is expressed in testicular, adrenal, and prostatic tumor tissues and is required for androgen biosynthesis.

CYP17 catalyzes two sequential reactions: 1) the conversion of pregnenolone and progesterone to their 17α-hydroxy derivatives by 17α-hydroxylase activity and 2) the subsequent formation of dehydroepiandrosterone (DHEA) and androstenedione, respectively, by C17,20 lyase activity. DHEA and androstenedione are androgens and are precursors of testosterone. Inhibition of CYP17 by abiraterone can also result in increased mineralocorticoid production by the adrenals [see Warnings and Precautions (5.1)].

Androgen sensitive prostatic carcinoma responds to treatment that decreases androgen levels. Androgen deprivation therapies, such as treatment with GnRH agonists or orchiectomy, decrease androgen production in the testes but do not affect androgen production by the adrenals or in the tumor.

ZYTIGA decreased serum testosterone and other androgens in patients in the placebo-controlled clinical trial. It is not necessary to monitor the effect of ZYTIGA on serum testosterone levels.

Changes in serum prostate specific antigen (PSA) levels may be observed but have not been shown to correlate with clinical benefit in individual patients.

### 12.3 Pharmacokinetics

Following administration of abiraterone acetate, the pharmacokinetics of abiraterone and abiraterone acetate have been studied in healthy subjects and in patients with metastatic CRPC. In vivo, abiraterone acetate is converted to abiraterone. In clinical studies, abiraterone acetate plasma concentrations were below detectable levels (<0.2 μg/mL) in >99% of the analyzed samples.

### Absorption

Following oral administration of abiraterone acetate to patients with metastatic CRPC, the median time to reach maximum plasma abiraterone concentrations is 2 hours. Abiraterone accumulation is observed at steady-state, with a 2-fold higher exposure (steady-state AUC) compared to a single 1,000 mg dose of abiraterone acetate.

At the dose of 1,000 mg daily in patients with metastatic CRPC, steady-state values (mean±SD) of Cₘₐₓ were 226±178 ng/mL and of AUC were 993±639 ng·hr/mL. No major deviation from dose proportionality was observed in the dose range of 250 mg to 1,000 mg. However, the exposure was not significantly increased when the dose was doubled from 1,000 to 2,000 mg (8% increase in the mean AUC).

Systemic exposure of abiraterone is increased when abiraterone acetate is administered with food. In healthy subjects abiraterone Cₘₐₓ and AUC_{0-∞} W were approximately 7- and 5-fold higher, respectively, when a single dose of abiraterone acetate was administered with a low-fat meal (7% fat, 300 calories) and approximately 17- and 10-fold higher, respectively, when a single dose of abiraterone acetate was administered with a high-fat (57% fat, 825 calories) meal compared to overnight fasting. Abiraterone AUC_{0-∞} was approximately 7-fold or 1.6-fold higher, respectively, when a single dose of abiraterone acetate was administered 2 hours after or 1 hour before a medium fat meal (25% fat, 491 calories) compared to overnight fasting.

Systemic exposures of abiraterone in patients with metastatic CRPC, after repeated dosing of abiraterone acetate were similar when abiraterone acetate was taken with low-fat meals for 7 days and increased approximately 2-fold when taken with high-fat meals for 7 days compared to when taken at least 2 hours after a meal and at least 1 hour before a meal for 7 days.

Given the normal variation in the content and composition of meals, taking ZYTIGA with meals has the potential to result in increased and highly variable exposures. Therefore, no food should be consumed for at least two hours before the dose of ZYTIGA is taken and for at least one hour after the dose of ZYTIGA is taken. The tablets should be swallowed whole with water [see Dosage and Administration (2.3)].

### Distribution and Protein Binding

Abiraterone is highly bound (>99%) to the human plasma proteins, albumin and alpha-1 acid glycoprotein. The apparent steady-state volume of distribution (mean±SD) is 19,669±13,358 L. In vitro studies show that at clinically relevant concentrations, abiraterone acetate and abiraterone are not substrates of P-glycoprotein (P-gp) and that abiraterone acetate is an inhibitor of P-gp.

### Metabolism

Following oral administration of ¹⁴C-abiraterone acetate as capsules, abiraterone acetate is hydrolyzed to abiraterone (active metabolite). The conversion is likely through esterase activity (the esterases have not been identified) and is not CYP mediated. The two main circulating metabolites of abiraterone in human plasma are abiraterone sulphate (inactive) and N-oxide abiraterone sulphate (inactive), which account for about 43% of exposure each. CYP3A4 and SULT2A1 are the enzymes involved in the formation of N-oxide abiraterone sulphate and SULT2A1 is involved in the formation of abiraterone sulphate.

### Excretion

In patients with metastatic CRPC, the mean terminal half-life of abiraterone in plasma (mean±SD) is 12±5 hours. Following oral administration of ¹⁴C-abiraterone acetate, approximately 88% of the radioactive dose is recovered in feces and approximately 5% in urine. The major compounds present in feces are unchanged abiraterone acetate and abiraterone (approximately 55% and 22% of the administered dose, respectively).

Patients with Hepatic Impairment

The pharmacokinetics of abiraterone was examined in subjects with baseline mild (N=8) or moderate (N=8) hepatic impairment (Child-Pugh Class A and B, respectively) and in 8 healthy control subjects with normal hepatic function. Systemic exposure to abiraterone after a single oral 1,000 mg dose given under tasting conditions increased approximately 1.1-fold and 3.6-fold in subjects with mild and moderate baseline hepatic impairment, respectively. The mean half-life of abiraterone is prolonged to approximately 18 hours in subjects with mild hepatic impairment and to approximately 19 hours in subjects with moderate hepatic impairment.

In another trial, the pharmacokinetics of abiraterone were examined in subjects with baseline severe (N=8) hepatic impairment (Child-Pugh Class C) and in 8 healthy control subjects with normal hepatic function. The systemic exposure (AUC) of abiraterone increased by approximately 7-fold in subjects with severe baseline hepatic impairment compared to subjects with normal hepatic function. In addition, the mean protein binding was found to be lower in the severe hepatic impairment group compared to the normal hepatic function group, which resulted in a two-fold increase in the fraction of free drug in patients with severe hepatic impairment [see Dosage and Administration (2.4) and Use in Specific Populations (8.6)].

Patients with Renal Impairment

The pharmacokinetics of abiraterone were examined in patients with end-stage renal disease (ESRD) on a stable hemodialysis schedule (N=8) and in matched control subjects with normal renal function (N=8). In the ESRD cohort of the trial, a single 1,000 mg ZYTIGA dose was given under fasting conditions 1 hour after dialysis, and samples for pharmacokinetic analysis were collected up to 96 hours post dose. Systemic exposure to abiraterone after a single oral 1,000 mg dose did not increase in subjects with end-stage renal disease on dialysis, compared to subjects with normal renal function [see Use in Specific Populations (8.7)].

### Drug Interactions

In vitro studies with human hepatic microsomes showed that abiraterone has the potential to inhibit CYP1A2, CYP2D6, CYP2C8 and to a lesser extent CYP2C9, CYP2C19 and CYP3A4/5.

In an in vivo drug-drug interaction trial, the Cₘₐₓ and AUC of dextromethorphan (CYP2D6 substrate) were increased 2.8- and 2.9-fold, respectively when dextromethorphan 30 mg was given with abiraterone acetate 1,000 mg daily (plus prednisone 5 mg twice daily). The AUC for dextrorphan, the active metabolite of dextromethorphan, increased approximately 1.3 fold [see Drug Interactions (7.2)].

In a clinical study to determine the effects of abiraterone acetate 1,000 mg daily (plus prednisone 5 mg twice daily) on a single 100 mg dose of the CYP1A2 substrate theophylline, no increase in systemic exposure of theophylline was observed.

Abiraterone is a substrate of CYP3A4, in vitro. In a clinical pharmacokinetic interaction study of healthy subjects pretreated with a strong CYP3A4 inducer (rifampin, 600 mg daily for 6 days) followed by a single dose of abiraterone acetate 1,000 mg, the mean plasma AUC_{∞} of abiraterone was decreased by 55% [see Drug Interactions (7.1)].

In a separate clinical pharmacokinetic interaction study of healthy subjects, co-administration of ketoconazole, a strong inhibitor of CYP3A4, had no clinically meaningful effect on the pharmacokinetics of abiraterone [see Drug Interactions (7.1)].

In a CYP2C8 drug-drug interaction trial in healthy subjects, the AUC of pioglitazone was increased by 46% when pioglitazone was given together with a single dose of 1,000 mg abiraterone acetate [see Drug Interactions (7.2)].

In vitro, abiraterone and its major metabolites were shown to inhibit the hepatic uptake transporter OATP1B1. There are no clinical data available to confirm transporter based interaction.

### 12.6 QT Prolongation

In a multi-center, open-label, single-arm trial, 33 patients with metastatic CRPC received ZYTIGA orally at a dose of 1,000 mg once daily at least 1 hour before or 2 hours after a meal in combination with prednisone 5 mg orally twice daily. Assessments up to Cycle 2 Day 2 showed no large changes in the QTc interval (i.e., >20 ms) front baseline. However, small increases in the QTc interval (i.e., <10 ms) due to abiraterone acetate cannot be excluded due to study design limitations.

### Nonclinical Toxicology

### 10.1 Carcinogenesis, Mutagenesis, and Impairment of Fertility

A two-year carcinogenicity study was conducted in rats at oral abiraterone acetate doses of 5, 15, and 50 mg/kg/day for males and 15, 50, and 150 mg/kg/day for females. Abiraterone acetate increased the combined incidence of interstitial, cell adenomas and carcinomas in the testes at all dose levels tested. This finding is considered to be related to the pharmacological activity of abiraterone. Rats are regarded as more sensitive than humans to developing interstitial cell tumors in the testes. Abiraterone acetate was not carcinogenic in female rats at exposure levels up to 0.8 times the human clinical exposure based on AUC. Abiraterone acetate was not carcinogenic in a 6-month study in the transgenic (Tg.rasH2) mouse.

Abiraterone acetate and abiraterone was not mutagenic in an in vitro microbial mutagenesis (Ames) assay or clastogenic in an in vitro cytogenetic assay using primary human lymphocytes or an in vivo rat micronucleus assay.

In repeat-dose toxicity studies in male rats (13- and 26-weeks) and monkeys (39-weeks), atrophy, aspermia/hypospermia, and hyperplasia in the reproductive system were observed at ≥50 mg/kg/day in rats and ≥250 mg/kg/day in monkeys and were consistent with the antiandrogenic pharmacological activity of abiraterone. These effects were observed in rats at systemic exposures similar to humans and in monkeys at exposures approximately 0.6 times the AUC in humans.

In a fertility study in male rats, reduced organ weights of the reproduction system, sperm counts, sperm motility altered sperm morphology and decreased fertility were observed in animals dosed for 4 weeks at ≥30 mg/kg/day orally. Mating of untreated females with males that received 30 mg/kg/day oral abiraterone acetate resulted in a reduced number of corpora lutea, implantations and live embryos and an increased incidence of pre-implantation loss. Effects on male rats were reversible after 16 weeks from the last abiraterone acetate administration.

In a fertility study in female rats, animals dosed orally for 2 weeks until day 7 of pregnancy at ≥30 mg/kg/day had an increased incidence of irregular or extended estrous cycles and pre-implantation loss (300 mg/kg/day). There were no differences in mating, fertility, and litter parameters in female rats that received abiraterone acetate. Effects on female rats were reversible after 4 weeks from the last abiraterone acetate administration.

The dose of 30 mg/kg/day in rats is approximately 0.3 times the recommended dose of 1,000 mg/day based on body surface area.

In 13- and 26-week studies in rats and 13- and 39-week studies in monkeys, a reduction in circulating testosterone levels occurred with abiraterone acetate at approximately one half the human clinical exposure based on AUC. As a result, decreases in organ weights and toxicities were observed in the male and female reproductive system, adrenal glands, liver, pituitary (rats only), and male mammary glands. The changes in the reproductive organs are consistent with the antiandrogenic pharmacological activity of abiraterone acetate.

Animal Toxicology and/or Pharmacology

A dose-dependent increase in cataracts was observed in rats after daily oral abiraterone acetate administration for 26 weeks starting at ≥50 mg/kg/day (similar to the human clinical exposure based on AUC). In a 39-week monkey study with dally oral abiraterone acetate administration, no cataracts were observed at higher doses (2 times greater than the clinical exposure based on AUC).

### Clinical Studies

The efficacy and safety of ZYTIGA with prednisone was established in three randomized placebo-controlled international clinical studies. All patients in these studies received a GnRH analog or had prior bilateral orchiectomy. Patients with prior ketoconazole treatment for prostate cancer and a history of adrenal gland or pituitary disorders were excluded from these trials. Concurrent use of spironolactone was not allowed during the study period.

COU-AA-301 (NCT00638690): Patients with Metastatic CRPC Who Had Received Prior Docetaxel Chemotherapy

A total of 1195 patients were randomized 2:1 to receive either ZYTIGA orally at a dose of 1,000 mg once daily in combination with prednisone 5 mg orally twice daily (N=797) or placebo once daily plus prednisone 5 mg orally twice daily (N=398). Patron is randomized to either arm were to continue treatment until disease progression (defined as a 25% increase in PSA over the patient's baseline/nadir together with protocol-defined radiographic progression and symptomatic or clinical progression), initiation of new treatment, unacceptable toxicity or withdrawal.

The following patient demographics and baseline disease characteristics were balanced between the treatment arms. The median age was 69 years (range 39-95) and the racial distribution was 93% Caucasian, 3.6% Black, 1.7% Asian and 1.6% Other. Eighty-nine percent of patients enrolled had an ECOG performance status score of 0-1 and 45% had a Brief Pain Inventory-Short Form score of ≥4 (patient's reported worst pain over the previous 24 hours). Ninety percent of patients had metastases in bone and 30% had visceral involvement. Seventy percent of patients had radiographic evidence of disease progression and 30% had PSA-only progression. Seventy percent of patients had previously received one cytotoxic chemotherapy regimen and 30% received two regimens.

The protocol pre-specified interim analysis was conducted after 552 deaths and showed a statistically significant improvement in overall survival (OS) in patients treated with ZYTIGA with prednisone compared to patients in the placebo with prednisone arm (Table 9 and FIG. 1). An updated survival analysis was conducted when 775 deaths (97% of the planned number of deaths for final analysis) were observed. Results from tins analysis were consistent with those from the interim analysis (Table 13).

TABLE 13
Overall Survival of Patients Treated with Either
ZYTIGA or Placebo in Combination with Prednisone
in COU-AA-301 (Intent-to-Treat Analysis)

| | ZYTIGA with | | Placebo with | |
|---|---|---|---|---|
| | Prednisone | | Prednisone | |
| | (N = 797) | | (N = 398) | |
| Primary Survival Analysis | | | | |
| Deaths (%) | 333 | (42%) | 219 | (55%) |
| Median survival (months) | 14.8 | (14.1, 15.4) | 30.9 | (30.2, 12.0) |
| (95% CI) | | | | |
| p-value¹ | <0.0001 | | | |
| Hazard ratio (95% CI)² | 0.646 (0.543, 0.768) | | | |
| Updated Survival Analysis | | | | |
| Deaths (%) | 501 | (63%) | 274 | (69%) |
| Median survival (months) | 15.8 | (14.8, 17.0) | 11.2 | (10.4, 13.1) |

(95% CI)

| | |
|---|---|
| Hazard ratio (95% CI)² | 0.740 (0.638, 0.859) |

¹p-value is derived from a log-rank test stratified by ECOG performance status score (0-1 vs. 2), pain score (absent vs. present), number of prior chemotherapy regimens (1 vs. 2), and type of disease progression (PSA only vs. radiographic).
²Hazard Ratio is derived from a stratified proportional hazards model. Hazard ratio <1 favors ZYTIGA with prednisone.

COU-AA-302 (NCT00887198): Patients with Metastatic CRPC Who Had not Received Prior Cytotoxic Chemotherapy

In COU-AA-302, 1088 patients were randomized 1:1 to receive either ZYTIGA orally at a dose of 1,000 mg once daily (N=546) or Placebo orally once daily (N=542). Both arms were given concomitant prednisone 5 mg twice daily. Patients continued treatment until radiographic or clinical (cytotoxic chemotherapy, radiation or surgical treatment for cancer, pain requiring chronic opioids, or ECOG performance status decline to 3 or more) disease progression, unacceptable toxicity or withdrawal. Patients with moderate or severe pain, opiate use for cancer pain, or visceral organ metastases were excluded.

Patient demographics were balanced between the treatment arms. The median age was 70 years. The racial distribution of patients treated with ZYTIGA was 95% Caucasian, 2.8% Black, 0.7% Asian and 1.1% Other. The ECOG performance status was 0 for 76% of patients, and 1 for 24% of patients. Co-primary efficacy endpoints were overall survival and radiographic progression-free survival (rPFS). Baseline pain assessment was 0-1 (asymptomatic) in 66% of patients and 2-3 (mildly symptomatic) in 26% of patients as defined by the Brief Pain Inventory-Short Form (worst pain over the last 24 hours).

Radiographic progression-free survival was assessed with the use of sequential imaging studies and was defined by bone scan identification of 2 or more new bone lesions with confirmation (Prostate Cancer Working Group 2 criteria) and/or modified Response Evaluation Criteria In Solid Tumors (RECIST) criteria for progression of soft tissue lesions. Analysis of rPFS utilized centrally-reviewed radiographic assessment of progression.

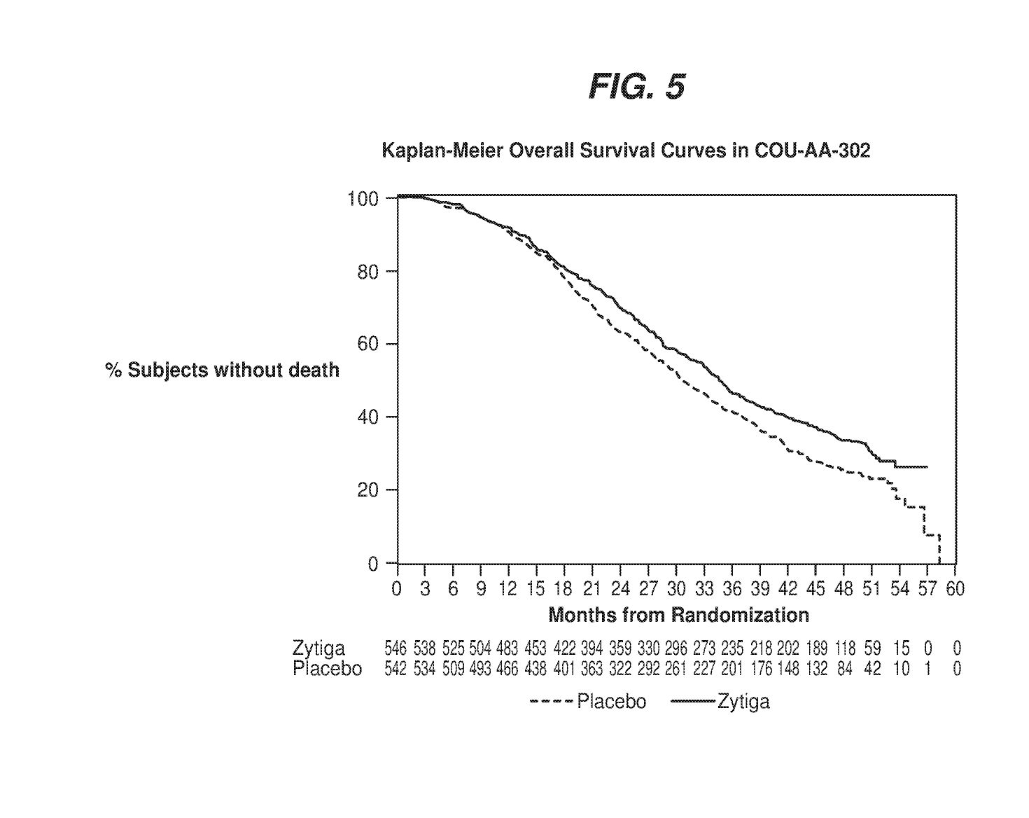

The planned final analysis for OS, conducted after 741 deaths (median follow up of 49 months) demonstrated a statistically significant OS improvement in patients treated with ZYTIGA with prednisone compared to those treated with placebo with prednisone (Table 14 and FIG. 5). Sixty-five percent of patients on the ZYTIGA arm and 78% of patients on the placebo arm used subsequent therapies that may prolong OS in metastatic CRPC ZYTIGA was used as a subsequent therapy in 13% of patients on the ZYTIGA arm and 44% of patients on the placebo arm.

TABLE 14
Overall Survival of Patients Treated with Either
ZYTIGA or Placebo in Combination with Prednisone
in COU-AA-302 (Intent-to-Treat Analysis)

| | ZYTIGA with | | Placebo with | |
|---|---|---|---|---|
| | Prednisone | | Prednisone | |
| Overall Survival | (N = 546) | | (N = 542) | |
| Deaths | 354 | (65%) | 387 | (71%) |
| Median survival (months) | 34.7 | (32.7, 36.8) | 30.3 | (28.7, 33.3) |
| (95% CI) | | | | |

| | |
|---|---|
| p-value¹ | 0.0033 |
| Hazard ratio² (95% CI) | 0.81 (0.70, 0.93) |

¹p-value is derived from a log-rank test stratified by ECOG performance status score (0 vs. 1).
²Hazard Ratio is derived from a stratified proportional hazards model. Hazard ratio <1 favors ZYTIGA with prednisone.

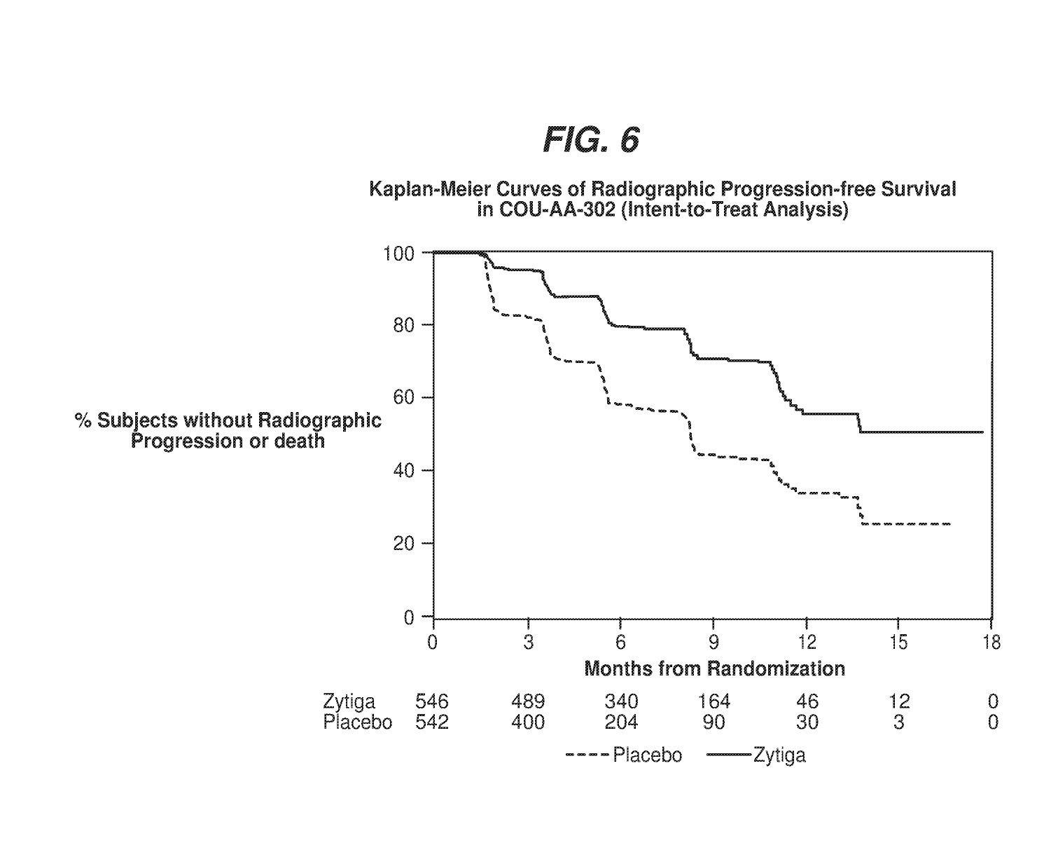

At the pre-specified rPFS analysis, 150 (28%) patients treated with ZYTIGA with prednisone and 251 (46%) patients treated with placebo with prednisone had radiographic progression. A significant difference In rPFS between treatment groups was observed (Table 15 and FIG. 6).

TABLE 15
Radiographic Progression-free Survival of Patients Treated
with Either ZYTIGA or Placebo in Combination with Prednisone
in COU-AA-302 (Intent-to-Treat Analysis)

| | ZYTIGA with | | Placebo with | |
|---|---|---|---|---|
| Radiographic Progression-free | Prednisone | | Prednisone | |
| Survival | (N = 546) | | (N = 542) | |
| Progression or death | 150 | (28%) | 251 | (46%) |
| Median rPFS (months) | NR | (11.66, NR) | 8.28 | (8.12, 8.54) |

(95% CI)

| | |
|---|---|
| p-value¹ | <0.0001 |
| Hazard ratio² (95% CI) | 0.425 (0.347, 0.522) |

NR = Not reached.
¹p-value is derived from a log-rank test stratified by ECOG performance status score (0 vs. 1).
²Hazard Ratio is derived from a stratified proportional hazards model. Hazard ratio <1 favors ZYTIGA with prednisone.

The primary efficacy analyses are supported by the following prospectively defined endpoints. The median time to initiation of cytotoxic chemotherapy was 25.2 months for patients in the ZYTIGA arm and 16.8 months for patients in the placebo arm (HR=0.580); 95% CI: [0.487, 0.691], p<0.0001).

The median time to opiate use for prostate cancer pain was not reached for patients receiving ZYTIGA and was 23.7 months for patients receiving placebo (HR=0.686; 95% CI: [0.566, 0.833], p=0.0001). The time to opiate use result was supported by a delay in patient reported pain progression favoring the ZYTIGA arm.

LATITUDE (NCT01715285); Patients with Metastatic High-Risk CSPC

In LATITUDE, 11.99 patients with metastatic high-risk CSPC were randomized 1:1 to receive either ZYTIGA orally at a dose of 1,000 mg once daily with prednisone 5 mg once daily (N=597) or placebos orally once daily (N=602). High-risk disease was defined as having at least two of three risk factors at baseline: a total Gleason score of ≥8, presence of ≥3 lesions on bone scan, and evidence of measurable visceral metastases. Patients with significant cardiac, adrenal, or hepatic dysfunction were excluded. Patients continued treatment until radiographic or clinical disease progression, unacceptable toxicity, withdrawal or death. Clinical progression was defined as the need for cytotoxic chemotherapy, radiation or surgical treatment for cancer, pain requiring chronic opioids, or ECOG performance status decline ≥3.

Patient demographics were balanced between the treatment arms. The median age was 67 years. The racial distribution of patients treated with ZYTIGA was 69% Caucasian, 2.5% Black, 21% Asian, and 8.1% Other. The ECOG performance status was 0 for 55%, 1 for 42%, and 2 for 3.5% of patients. Baseline pain assessment was 0-1 (asymptomatic) in 50% of patients, 2-3 (mildly symptomatic) in 23% of patients, and ≥4 in 28% of patients as defined by the Brief Pain Inventory-Short Form (worst pain over the last 24 hours).

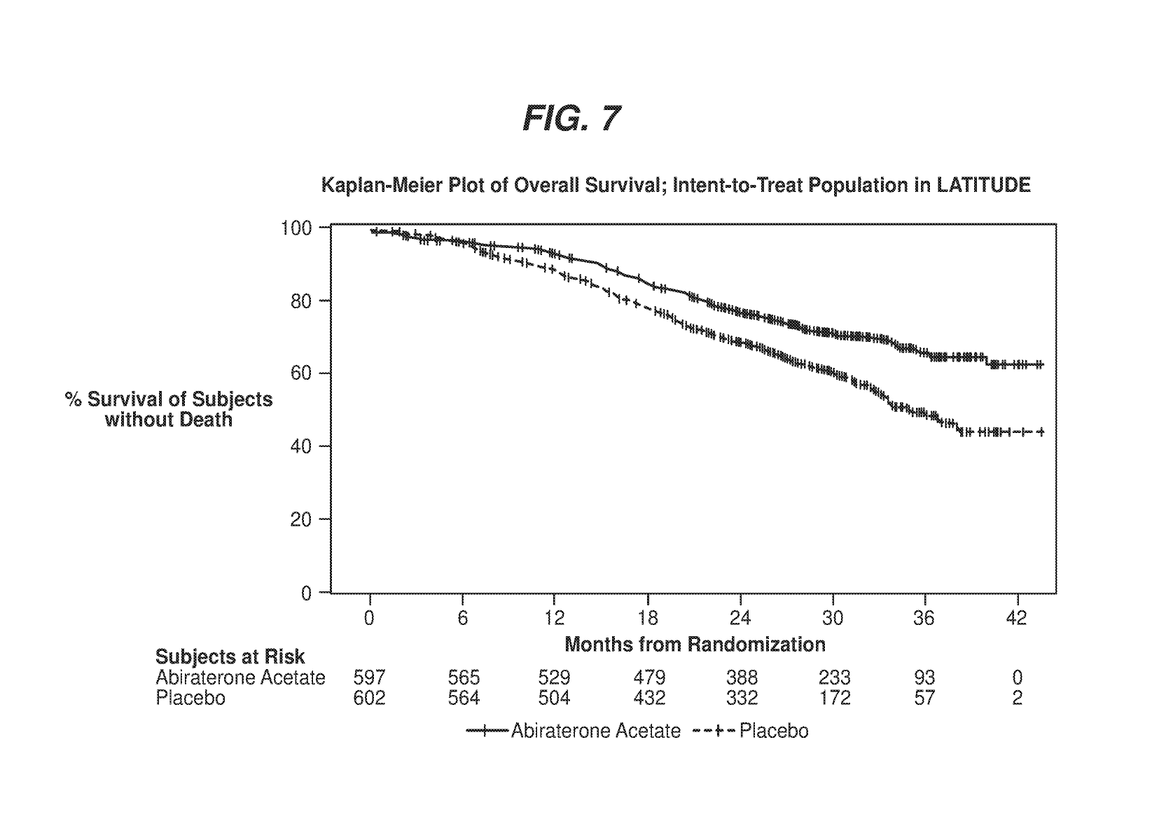

A major efficacy outcome was overall survival. The pre-specified interim analysis was conducted after 406 deaths and showed a statistically significant improvement in OS in patients on ZYTIGA with prednisone compared to those on placebos (see Table 16 and FIG. 7). Twenty-one percent of patients on the ZYTIGA arm and 41% of patients on the placebo arm received subsequent therapies that may prolong OS in metastatic CRPC, including cytotoxic chemotherapy, abiraterone acetate, enzalutamide, and systemic radiotherapy.

TABLE 16
Overall Survival of Patients Treated with Either
ZYTIGA in Combination with Prednisone or Placebos
in LATITUDE (Intent-to-Treat Analysis)

| | ZYTIGA with | | |
|---|---|---|---|
| | Prednisone | Placebos | |
| Overall Survival | (N = 597) | (N = 602) | |
| Deaths | 169 (28%) | 237 | (39%) |
| Median survival (months) | NE | 34.7 | (33.1, NE) |
| (95% CI) | | | |

| | |
|---|---|
| p-value¹ | <0.0001 |
| Hazard ratio² (95% CI) | 0.621 (0.509, 0.756) |

NE = Not estimable.
¹p value is from log-rank test stratified by ECOG PS score (0/1 or 2) and visceral (absent or present)
²Hazard Ratio is derived from a stratified proportional hazards model. Hazard ratio <1 favors ZYTIGA with prednisone.

The major efficacy outcome was supported by a statistically significant delay in time to initiation of chemotherapy for patients in the ZYTIGA arm compared to those in the placebos arm. The median time to initiation of chemotherapy was not reached for patients on ZYTIGA with prednisone and was 38.9 months for patients on placebos (HR=0.44; 95% CI: [0.35, 0.56], p<0.0001).

### 16 How Supplied/Storage and Handling

ZYTIGA® (abiraterone acetate) Tablets are available in the strengths and packages listed below:
ZYTIGA® 500 mg film-coated Tablets
   Purple, oval-shaped tablets debossed with “AA” one side and “500” on the other side.
   NDC 57894-195-06 60 tablets available in high-density polyethylene bottles
ZYTIGA® 256 mg film-coated Tablets
   Pink, oval-shaped tablets debossed with “AA250” on one side,
   NDC 57894-184-12 120 tablets available in high-density polyethylene bottles
ZYTIGA® 250 mg uncoated Tablets
   White to off-white, oval-shaped tablets debossed with “AA250” on one side,
   NDC 57894-150-12 120 tablets available in high-density polyethylene bottles

### Storage and Handling

Store at 20° C. to 25° C. (68° F. to 77° F.); excursions permitted in the range from 15° C. to 30° C. (59° F. to 86° F.) [see USP Controlled Room Temperature].

Keep out of reach of children.

Based on its mechanism of action, ZYTIGA may harm a developing fetus. Women who are pregnant or women who may be pregnant should not handle ZYTIGA 250 mg uncoated tablets or other ZYTIGA tablets if broken, crushed, or damaged without protection, e.g., gloves [see Use in Specific Populations (8.1)].

### Patient Counseling Information

Advise the patient to read the FDA-approved patient labeling (Patient Informal ion)

### Hypertension, Hypokalemia, and Fluid Retention

Inform patients that ZYTIGA is associated with hypertension, hypokalemia, and peripheral edema. Advise patients to report symptoms of hypertension, hypokalemia, or edema to their healthcare provider [see Warnings and Precautions (5.1)].

### Adrenocortical Insufficiency

Inform patients that ZYTIGA with prednisone is associated with adrenal insufficiency. Advise patients to report symptoms of adrenocortical insufficiency to their healthcare provider [see Warnings and Precautions (5.2)].

### Hepatotoxicity

Inform patients that ZYTIGA is associated with severe hepatotoxicity. Inform patients that their liver function will be monitored using blood tests. Advise patients to immediately report symptoms of hepatotoxicity to their healthcare provider [see Warnings and Precautions (5.3)].

### Dosing and Administration

Inform patients that ZYTIGA is taken once daily with prednisone (once or twice daily according to their healthcare provider's instructions) and to not interrupt or stop either of these medications without consulting their healthcare provider.
Inform patients receiving GnRH therapy that they need to maintain this treatment during the course of treatment with ZYTIGA.
Instruct patients to take ZYTIGA on an empty stomach, either one hour before or two hours after a meal ZYTIGA taken with food causes increased exposure and may result in adverse reactions. Instruct patients to swallow tablets whole with water and not to crush or chew the tablets [see Dosage and Administration (2.3)].
Inform patients that if they miss a dose of ZYTIGA or prednisone, they should take their normal dose the following day. If more than one daily dose is skipped, inform patients to contact their healthcare provider [see Dosage and Administration (2.3)].

### Fetal Toxicity

Inform patients that ZYTIGA may harm a developing fetus. Advise males with female partners of reproductive potential to use effective contraception during treatment and for 3 weeks after the final dose of ZYTIGA [see Use in Specific Populations (8.1)].
Women who are pregnant or women who may be pregnant should not handle ZYTIGA 250 mg uncoated tablets or other ZYTIGA tablets if broken, crushed, or damaged without protection, e.g., gloves [see Use in Specific Populations (8.1) and How Supplied/Storage and Handling (16)].

### Infertility

Advise male patients that ZYTIGA may impair fertility [see Use in Specific Populations (8.3)].
500 mg Tablets
Manufactured by:
Patheon France S.A.S.
Bourgoin Jallieu, France
250 mg Tablets
Manufactured by:
Patheon Inc.
Mississauga, Canada
Manufactured for:
Janssen Biotech, Inc.
Horsham, PA 19044
©2011, 2017 Janssen Pharmaceutical Companies
Revised: February 2018

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method of treating metastatic high-risk castration-sensitive prostate cancer in a human male comprising adding a safe and effective amount of abiraterone acetate and a safe and effective amount of prednisone to androgen deprivation therapy in such human.

2. A method of claim 1, comprising administering to the human about 1000 mg/day of abiraterone acetate and about 5 mg/day of prednisone.

3. A method of claim 1, wherein the androgen deprivation therapy comprises an orchiectomy.

4. A method of claim 1, wherein the androgen deprivation therapy comprises a hormonal ablation agent that is selected from the group consisting of histrelin acetate, leuprolide acetate, goserelin acetate, and triptorelin palmoate.

5. A method of treating newly diagnosed metastatic high-risk castration-sensitive prostate cancer in a human male comprising administering to such human a combination therapy demonstrated to increase overall survival of men with newly diagnosed metastatic castration-sensitive prostate cancer, wherein the treatment comprises safe and effective amounts of abiraterone acetate, prednisone and androgen deprivation therapy.

6. A method of treating newly diagnosed metastatic high-risk castration-sensitive prostate cancer in a human male comprising administering to such human a combination therapy demonstrated to increase radiographic progression-free survival of men with newly diagnosed metastatic castration-sensitive prostate cancer, wherein the treatment comprises safe and effective amounts of abiraterone acetate, prednisone and androgen deprivation therapy.

7. A method of treating a newly diagnosed metastatic high-risk castration-sensitive prostate cancer in a human male comprising administering to such human a combination therapy demonstrated to increase time to pain progression for men with newly diagnosed metastatic castration-sensitive prostate cancer, wherein the treatment comprises safe and effective amounts of abiraterone acetate, prednisone and androgen deprivation therapy.

8. A method of treating a newly diagnosed metastatic high-risk castration-sensitive prostate cancer in a human male comprising administering to such human a combination therapy demonstrated to increase time to a next symptomatic skeletal event for men with newly diagnosed metastatic castration-sensitive prostate cancer, wherein the treatment comprises safe and effective amounts of abiraterone acetate, prednisone and androgen deprivation therapy.

9. A method of treating new diagnosed metastatic high-risk castration-sensitive prostate cancer prostate cancer in a human male, comprising administering such human a combination therapy demonstrated to increase time to time to PSA progression for men with newly diagnosed metastatic castration-sensitive prostate cancer, wherein the treatment comprises safe and effective amounts of abiraterone acetate, prednisone and androgen deprivation therapy.

10. A method of selling a drug product comprising abiraterone acetate, said method comprising selling such drug product, wherein a drug product label for a reference listed drug for abiraterone acetate includes an indication for treating patients with metastatic high-risk castration-sensitive prostate cancer using abiraterone acetate in combination prednisone.

11. The method of claim 10, wherein the drug product is an ANDA drug product or a supplemental New Drug Application drug product.

12. A method of selling a drug product comprising abiraterone acetate, said method comprising selling such drug product, wherein a drug product label for a reference listed drug for abiraterone acetate includes data from LATITUDE: patients with metastatic high-risk castration-sensitive prostate cancer.

13. The method of claim 12, wherein the drug product is an ANDA drug product or a supplemental New Drug Application drug product.

14. A method of claim 12, wherein the drug product label comprises a hazard ratio of 0.621 for overall survival.

15. The method of claim 14, wherein the drug product is an ANDA drug product or a supplemental New Drug Application drug product.

16. The method of offering for sale a drug product comprising abiraterone acetate, said method comprising offering for sale such drug product, wherein the drug product label for a reference listed for abiraterone acetate includes an indication for the treatment of patients with metastatic high-risk castration-sensitive prostate cancer with abiraterone acetate in combination with prednisone

17. The method of claim 16, wherein the drug product is an ANDA drug product or a supplemental New Drug Application drug product.

18. A method of selling a drug product comprising abiraterone acetate, said method comprising selling such drug product, wherein a drug product label for a reference listed drug for abiraterone acetate includes data from LATITUDE: patients with metastatic high-risk castration-sensitive prostate cancer.

19. The method of claim 18, wherein the drug product is an ANDA drug product or a supplemental New Drug Application drug product.

20. A method of claim 18, wherein the drug product label comprises a hazard ratio of 0.621 for overall survival.

21. The method of claim 19, wherein the drug product is an ANDA drug product or a supplemental New Drug Application drug product.

22. A method of improving overall survival in a male human with metastatic high-risk castration-sensitive prostate cancer, said method comprising providing to said male human an approved drug product comprising abiraterone acetate and prednisone in combination with androgen deprivation therapy.

23. The method of claim 22, wherein the overall survival is was not estimable with a 95% confidence interval.

24. The method of claim 22 or 23, wherein the approved drug product is an ANDA drug product or a supplemental New Drug Application drug product.

25. The method of any one of claims 22 to 24, wherein the drug product label for a reference listed drug for such drug product includes an indication for patients with metastatic high-risk castration-sensitive prostate cancer using abiraterone acetate and prednisone.
